(19) United States
(12) Patent Application Publication
(10) Pub. No.: US 2015/0030681 A1
(43) Pub. Date: Jan. 29, 2015

(54) METHOD OF MAKING A HYDROGEL

(71) Applicant: The University of Manchester, Manchester (GB)

(72) Inventors: Catherine Louise Ruby Merry, High Peak (GB); Alberto Saiani, Prestbury (GB); Kate Alexandra Meade, Manchester (GB); Emma Tranquility Lowe, Reddish (GB); Aline Fiona Saiani, Prestbury (GB); Jean-Baptiste Guilbaud, Liverpool (GB)

(73) Assignee: The University of Manchester, Manchester (GB)

(21) Appl. No.: 14/379,911

(22) PCT Filed: Feb. 8, 2013
(86) PCT No.: PCT/GB2013/050295

§ 371 Date: Aug. 20, 2014

(30) Foreign Application Priority Data
Feb. 22, 2012 (GB) 1203085.4

(51) Int. Cl. C12N 5/00; C12N 5/0775; C12N 15/10; C12N 5/0735

(52) U.S. Cl. CPC C12N 5/0012; C12N 5/0606; C12N 5/0663; C12N 15/1003; C12N 2533/50
USPC 424/491; 435/177; 530/412; 536/25.41; 424/93.7; 506/26; 435/397

(57) ABSTRACT

The present invention relates to a novel protocol for making a hydrogel, which shows increased stability compared to hydrogels of the art, and can be reliably reproduced. The hydrogels produced by the methods of the present invention are preferably three dimensional, and particularly suitable for the culture of stem cells.

The present invention relates to a method of making a hydrogel. In particular, the present invention relates to a method of encapsulating cells in a hydrogel. The invention also relates to a method of culturing cells in a hydrogel, a method of extracting cells from a hydrogel, and a method of tissue regeneration using a hydrogel. The present invention also provides a hydrogel, made by the method of the invention; and a kit for making a hydrogel of the present invention.

### BACKGROUND

Hydrogels are hydrated, porous materials, which have utility in cell culture techniques, as well as in tissue engineering. Three-dimensional hydrogels are particularly useful in cell applications, for example for growing cells for tissue engineering or tissue regeneration, and studying cells to analyse their behaviour, for example in terms of growth and differentiation and response to external factors such as stress. To maximise the potential of hydrogels for in vitro cell culture, much research has been applied to developing hydrogels which mimic the natural extracellular matrix, and so are better able to support cell survival and growth. Biologically, hydrogels must satisfy a demanding set of criteria in order to be deemed suitable for cell culture or tissue regeneration techniques. For example, they must not be cytotoxic to cells; must be biocompatible (i.e. will not provide a significant immune response in vivo); and preferably will be biodegradable.

Typically, three-dimensional hydrogels are based upon a scaffold generated from amphiphilic molecules, for example peptides which have alternating hydrophilic and hydrophobic amino acids (see, for example, US2007/0099840). Amphiphilic molecules are advantageous in the production of three dimensional biological scaffolds because they have the ability to self-assemble into an organised structure, within which cells can be cultured.

Stem cells are of great interest to those working in biological and clinical fields because they have the ability to self renew and also to differentiate into a plurality of cell types. This means that they offer enormous potential for tissue engineering, drug/toxicity screening and other research and clinical applications. To harness this potential, the environmental requirements for their survival, expansion and differentiation in culture need to be better understood. The unique and cell type/line specific nature of the requirements to maintain them in culture adds further to the requirements a hydrogel must fulfil.

Whilst hydrogels have been known for culturing stem cells for some time, many difficulties still exist. Peptide based hydrogels include those which utilise a 16-mer amphiphilic peptide. These larger peptides significantly add to the cost of the hydrogel. Other hydrogels for cell culture are based upon animal- or bacteria-derived hyaluronic acid which is chemically cross linked to form the hydrogels. MAPTrix™ hydrogels are based upon recombinant expression of muscle adhesive protein with the addition of short peptide motifs from extracellular matrix proteins which increase stem cell attachment.

Commercially available hydrogels have been slow to be accepted for cell culture and growth, especially for stem cells because in general they are difficult to use.

A significant problem is the lack of reliability, meaning that the chance of a peptide solution not setting into a hydrogel is unacceptably high, especially for stem cell applications where gel to gel passage is often desired. WO2002/062969 describes the preparation of peptide hydrogels, in particular hydrogels which encapsulate cells. The methodology comprises maintaining the cells and peptide in an iso-osmotic solution such as sucrose, which prevents gelation. Subsequently an electrolyte is added to the solution, which triggers gelation. In a similar vein, WO2006/014570 describes a method for producing a hydrogel having cells encapsulated therein. The gel is formed by exposure of a peptide solution to a salt solution, which triggers gelation. US2002/0160471 describes a method of producing peptide hydrogel scaffolds, which comprises incubating peptides and live cells in an iso-osmotic solution, such that the peptides do not self-assemble, and then adding sufficient electrolyte to initiate gel formation, thereby encapsulating the cells within the gel as it forms. In all of these methods, the gel is formed for the first time with the cells inside, in order that they be encapsulated. Thus, if the gel does not form properly for any reason, as is often the case, the cells are sacrificed. The loss of cells at this stage can represent a significant loss in time, effort and resources in both research, commercial and medical applications.

WO2007/059491 describes the production of peptide based hydrogels, using 16-20mer amino acid chains which are amphiphilic in nature. The methodology described includes raising the pH of a peptide solution, thus causing assembly of the peptides into a higher order structure. As raising the pH is detrimental to cell viability, a gel formed in this way does not encapsulate the cells, but rather the cells are cultured on the surface of the pre-formed gel. This is less suited to most cell culture applications. Alternative methods described include adding buffer or media to a peptide solution, to cause gelation. The latter method is described for forming hydrogels with cells encapsulated therein. Again however, as it is variable whether or not the gel will form, the methodology risks the loss of cells if the gel does not properly form.

Thus, the methods of making peptide based hydrogels which encapsulate cells for culture are typically unreliable, and the chance of a gel not setting properly is high. Gels are also difficult to reliably reproduce, which means that gel to gel passage in which multiple gels of the same type are required, is difficult. Thus, to harness the potential of hydrogels for in vitro culture of stem cells, and their possible use in in vivo tissue regeneration or therapy, hydrogels are needed which can be more reliably produced, with reduced risk of cell sacrifice, and where the methodology can be more accurately reproduced.

The present invention aims to ameliorate or overcome some or all of the problems associated with the prior art, by providing a novel protocol for the preparation of a hydrogel.

### BRIEF SUMMARY OF THE DISCLOSURE

In a first aspect of the present invention, there is provided a method of making a hydrogel, comprising the steps of:
1) providing a peptide solution, wherein the peptide comprises an amphiphilic portion;
2) altering a characteristic of the peptide solution, wherein the characteristic is ionic strength, pH, temperature, or ion concentration, to form a first solution;
3) maintaining the first solution of 2) under conditions suitable for the formation of a hydrogel;
4) liquefying the hydrogel of 3);
5) combining the liquefied hydrogel of 4) with i) cells and optionally ii) media, to form a second solution; and
6) maintaining the second solution of 5) under conditions suitable for the formation of a hydrogel comprising cells encapsulated therein.

In an embodiment, the peptide is dissolved in a suitable solvent, preferably water.

In an embodiment, the characteristic of step 2) is altering the pH. Preferably, the pH is increased, preferably to between 8 and 10, preferably to between 8.8 and 9.5, preferably to between 9 and 9.5.

In an embodiment, the pH is increased by mixing the peptide solution with a base.

In an embodiment, a test of whether the first solution will form a gel is optical transparency of the first solution, upon addition of base. Preferably, a first solution which is capable of forming a hydrogel will be optically transparent between pH 8 and 10, upon addition of base.

Thus, in an embodiment of the first aspect of the invention, step 2) comprises determining the optical transparency of the first solution, and if not optically transparent, adding additional base until optical transparency is achieved. Preferably, the first solution is at a pH between 8 and 10, more preferably between 8.8 and 9.5, more preferably between 9 and 9.5. The optical transparency is preferably determined at a pH above pH 7.

The characteristic altered to form a gel at step 2) of the method is not required to be cell-compatible (i.e. cell friendly, meaning the method is not detrimental to the viability of any cells to be encapsulated). Thus, the method may be cell compatible or non-compatible. The former is preferred. Altering the ionic strength, temperature, or ion concentration may be performed by methods known in the art, for example addition of electrolyte, salt, or incubation in an oven or water bath. Salts include KCL, NaCL, MgCl₂, KF, and MgSO₄. Preferred salt concentrations are 200 mM to 400 mM. Ions include Ca²+.

In a preferred embodiment of the first aspect, step 1) of the method comprises the steps of:

i) determining a desired final volume of the gel;

ii) determining a desired final peptide concentration of the gel;

iii) calculating the mass of peptide to be used as final volume (ml)×final concentration (mg/ml);

iv) where the peptide is in dry form, providing it as a peptide solution;

v) mixing a base with the peptide solution of iv) to produce an optically transparent first solution.

In a preferred embodiment, base is mixed with the peptide solution to form a hydrogel. Preferably, the mixing of base and peptide takes place at a temperature between 60° C. and 95° C. In an embodiment, it is a continuous step following on from dissolving of the peptide. Alternatively, a peptide solution which has been previously prepared and/or stored may be used.

In an embodiment, the pH of the peptide solution is increased until the pH is between 8 and 10, and the first solution is optically transparent. Preferably, the gel formed by increasing the pH until the first solution is optically transparent. In an embodiment, the pH is increased by mixing the peptide solution with base. Sufficient base is used to achieve an optically transparent first solution and a pH of between 8 and 10, preferably to between 8.8 and 9.5, preferably to between 9 and 9.5.

In an embodiment, the hydrogel formed in step 3 is homogenous.

In a preferred embodiment of the first aspect, step 1) of the method further comprises the step of adding a buffer solution. It may be mixed with the peptide or with the first solution. Preferably, a buffer is added at step 2), before, after or with the base. Preferably, the addition of buffer does not alter the optical transparency. Any suitable buffer may be used. In an embodiment, the buffer is PBS.

In a preferred embodiment of the first aspect, the first solution is maintained under conditions suitable for allowing its transition into a hydrogel. Suitable conditions are between 20° C. and 37° C., preferably between 20 and 30° C., more preferably at between 20 and 26° C., typically between 21 and 23° C., for at least 20 minutes.

Step 3) of the method comprises liquefying the gel formed in step 3), using any suitable method. Mechanical agitation or other physical methods are preferred, although other methods, such as chemical methods, may be used. Any such methods preferably do not decrease the cell compatibility of the liquefied gel.

In an embodiment, the liquefied gel has pH of between 8 and 10, and is optically transparent. Preferably, the pH is between 8.8 and 9.5, more preferably between 9 and 9.5. Preferably, the pH of the liquefied gel is a cell-tolerant pH, preferably a pH which optimises cell culture whilst maintaining and maximising hydrogel stability. Preferably, the pH is that at which cells, preferably stem cells, are tolerant, preferably between 6.5 and 10.5, more preferably between pH 7 and 9.5, more preferably between pH 9 and 9.5.

In a preferred embodiment of the first aspect, step 4) of the method comprises the steps of:

i) calculating the amount of media for addition to the liquefied gel of 3) to provide media:liquefied gel ratio of between 1:1 and 1:10, preferably between 1:2 and 1:5 more preferably 1:4;

ii) mixing media with the liquefied gel until an optically transparent second solution is formed.

The ratio of liquefied gel to media is critical to the stability and reproducibility of the hydrogel, as it affects the buffering capacity, the effectiveness of cell encapsulation, and the stiffness of the gel. Suitable ratios can be determined by the person skilled in the art by altering the ratio, mixing media with the liquefied gel to produce a second solution which is preferably optically transparent, and assessing cell viability and cell culture, as described herein. Optimal ratios for media:liquefied gel have been found to be between 1 part media to 10 parts liquefied gel, preferably between 1 part media to 2 parts liquefied gel to 1 part media to 5 parts liquefied gel (1:2 to 1:5), more preferably 1 part media to 4 parts liquefied gel (1:4). Media can be added with the cells, or before or after the cells. It may be added step wise or all in one go.

Preferably, the temperature of the liquefied gel prior to addition of the media is between approximately 18 and 37° C., preferably between 20 and 30° C., more preferably at between 20 and 26° C., typically between 21 and 23° C. Preferably, after mixing with base at between 60 and 95° C., the resulting first solution is allowed to cool at room temperature.

Step 5) of the method comprises mixing the liquefied gel of 4) with i) cells which are to be encapsulated. Optionally, cells and media are provided. In a preferred embodiment, step 5) comprises adding cells before, after, or at the same time as media. The cells may be provided in the media. Upon maintenance under conditions suitable for the formation of a gel, the peptides self-assemble into a three dimensional structure to form a gel. The conditions under which the second solution is maintained to form a gel will preferably be cell-compatible, so as not to be detrimental to the viability of the cells encapsulated therein. There may be embodiments where cell viability is not critical, and non-compatible conditions may be used.

In an embodiment, a test of whether the second solution will form a gel is optical transparency. Preferably, a second solution which is capable of forming a hydrogel will be optically transparent. Preferably, the pH of the second liquid is cell-compatible, for example between 6.5 and 10.5, more preferably between 7 and 9.5, more preferably between 9 and 9.5. The optical transparency is preferably determined at a pH above pH 7.

Thus, in an embodiment of the first aspect of the invention, step 5) further comprises determining the optical transparency of the second liquid. If not optically transparent, the solution is discarded. Preferably, the second liquid is at a pH between 6.5 and 10.5.

In a preferred embodiment of the first aspect, the second solution is maintained under conditions suitable for its transition into a hydrogel. Suitable conditions are between 20° C. and 37° C., preferably between 20 and 30° C., more preferably at between 20 and 26° C., typically between 21 and 23° C., for at least 20 minutes.

In an embodiment, the first and second solutions are isotonic.

It is appreciated, however, that the pH of the first solution of 1), second solution and the final gel will depend upon the exact nature of peptides used, the synthetic route used to synthesise the peptide, the presence or absence of any functional groups, the type of media used, and the type and number of cells, etc. Thus, the pH at each step may vary. It is envisaged that in certain embodiments of the present invention, the pH may fall outside of the above mentioned range. Thus, the optical transparency of the first solution, second solution and final gel may be used as a test of suitability to produce a stable, reproducible hydrogel.

Mixing may be performed by any suitable means, for example stirring, shaking, pipetting, sonication etc. Other methods will be known to persons skilled in the art.

In a second aspect of the present invention, there is provided a method of making a hydrogel, the method comprising the steps of:

1) mixing a base with a solution of peptide comprising an amphiphilic portion to produce an optically transparent first solution;

2) maintaining the solution of 1) under conditions suitable for formation of a hydrogel;

3) optionally mixing the hydrogel of 2) with media to produce an optically transparent second solution;

4) maintaining the second solution under conditions suitable for the formation of a hydrogel.

In this aspect, the steps of liquefying and cell addition may be combined, and performed in a single mixing step.

Preferably, the first solution is at a pH between 8 and 10, more preferably between 8.8 and 9.5, more preferably between 9 and 9.5. Preferably, the pH of the second solution is cell-compatible, for example between 6.5 and 10.5, more preferably between 7 and 9.5, more preferably between 9 and 9.5.

Preferably, the preferred embodiments of the first aspect apply to the second aspect of the invention, mutatis mutandis.

In a third aspect of the invention, there is provided a method of encapsulating cells in a hydrogel, comprising:—
1) providing a pre-formed hydrogel;
2) liquefying the hydrogel;
3) combining the liquefied hydrogel of 2) with i) cells and optionally ii) media to form a second solution;
4) maintaining the second solution of 3) under conditions suitable for the formation of a hydrogel.

In an embodiment, the pre-formed hydrogel is prepared by steps 1) to 2) of the first aspect.

In an embodiment, the second solution is optically transparent. Preferably, the pH of the second liquid is cell-compatible, for example between 6.5 and 10.5, more preferably between 7 and 9.5, more preferably between 9 and 9.5.

The pre-formed hydrogel may have been previously frozen and defrosted, or stored at about 4° C.

Preferably, the preferred embodiments of the first and second aspects apply to the third aspect of the invention, mutatis mutandis.

In a fourth aspect of the invention, there is provided a method of culturing cells, the method comprising:

1) encapsulating cells in a hydrogel, prepared according to a method of the invention;

2) maintaining the hydrogel under biologically acceptable cell culture conditions for a cell culture period.

In a preferred embodiment, the cell culture period may be at least 1 hour, 2, 3, 4, 5, l 6, 7, 8, 9, 10, 12, 15, 20 or 24 hours, or at least 1, 2, 3, 4, 5, 6, 7, 8, 9, 10, 11, 12, 13, 14, 21, 25, 28, or at least 31 days.

In a preferred embodiment, a hydrogel is washed for example during the cell culture period. Thus, a method of the invention may further comprise the step of washing the hydrogel with fresh media one or more times during a cell culture period.

Preferably, the hydrogel is washed with fresh media an optimum number of times. The optimum number of washes is that which maximises one or more of i) cell viability; ii) cell growth; and iii) hydrogel stability.

In a preferred embodiment, the optimum frequency of washes may be determined by a method comprising the steps of:

i) preparing a plurality of hydrogels encapsulating a cell, according to a method of the invention;

ii) bringing a predetermined volume of media into contact with the gel;

iii) dividing the plurality of hydrogels into two or more batches;

iii) for a first batch of hydrogels, washing with media of ii) at a first predetermined frequency; for a second batch of gels, washing with media of ii) at a second predetermined frequency; and for any further batch of gels, washing with media of ii) at a further predetermined frequency;

iv) extracting any cells from the gel;

v) counting the cells and determining the optimal number of washes required to enhance, maximise or optimise one or more of i) cell survival; ii) cell growth; and iii) hydrogel stability within a hydrogel, compared to the cell survival, cell growth or hydrogel stability of a gel which undergoes an alternative frequency of washes.

Preferably, the preferred embodiments of the first, second and third aspects apply to the fourth aspect of the invention, mutatis mutandis.

In a fifth aspect of the invention, there is provided a method of extracting cells and/or cell-derived material from a hydrogel of the invention, the method comprising:
1) disrupting a hydrogel comprising cells prepared according to a method of invention, to fully or partially liquefy the gel;
2) separating any cells from the hydrogel or liquefied hydrogel;
3) optionally washing the cells.

Preferably, the method comprises first encapsulating a cell in a hydrogel according to the second aspect, and/or culturing a cell in a hydrogel according to the third aspect.

The gel may be liquefied by mechanical methods, or by chemical methods, for example as described herein.

In a preferred embodiment, an extracted cell of the fifth aspect may be encapsulated in a second or further hydrogel, preferably using a method of the third or fourth aspect. In a preferred embodiment, this method may comprise:

i) mixing an extracted cell with media;

ii) introducing the media comprising a cell into a second or further hydrogel, preferably using a method of the third aspect.

Preferably, step ii) comprises steps 1) and 2) of the third aspect.

In an embodiment, nucleic acids including RNA or DNA, proteins, sugars or other cellular material may be extracted from the cells.

Preferably, the preferred embodiments of the first, second, third and fourth aspects apply to the fifth aspect of the invention, mutatis mutandis.

In a sixth aspect of the invention, there is provided a method of tissue repair in a subject, comprising:

i) liquefying a hydrogel comprising a cell, wherein the hydrogel is prepared according to a method of the invention;

ii) introducing the liquefied hydrogel comprising a cell of i) into an appropriate tissue of a subject.

Under suitable in vivo biological conditions, the liquefied hydrogel comprising the cell sets into a hydrogel, thus adopting a shape dictated by its location within a subject.

In a preferred embodiment of the sixth aspect, mechanical disruption is used to liquefy the hydrogel.

Preferably, the preferred embodiments of the first to fifth aspects apply to the sixth aspect of the invention, mutatis mutandis.

Optimisation of the methods of making a hydrogel according to the present invention may comprise determining the amount of base to be added to the peptide. This method comprises the steps of:
i. determining the final volume of the gel to be made;
ii. determining the final concentration of the gel;
iii. calculating the amount of peptide to be used as volume (m/l) and final concentration;
iv. mixing the peptide of ii) or iii) with the base to produce a first solution;
v. determining the optical transparency of the first solution;
vi. if the first solution is not optically transparent, continuing to add base until an optically transparent solution is obtained;
vii. maintaining the solution under conditions suitable for formation of a hydrogel;
viii. assessing gel stability; wherein if unstable, discarding and repeating steps i) to vi) wherein a reduced amount of base is added in step iv);
ix. calculating the total amount of base added per mg of peptide used to obtain an optically transparent first solution and a stable hydrogel.

Optionally, the following steps may be followed:
i) where the gel is not optically transparent or is not stable, increasing the amount of base in step iii) by between 0.3 and 10% of the final gel volume until an optically transparent solution is obtained;
ii) where the gel is optically transparent but unstable, reducing the amount of base in step iii) until the gel is stable.

In a seventh aspect of the invention, there is provided a hydrogel which has one or more of the following independently selected properties:

i) is capable of a liquid-gel-liquid transition or a gel-liquid-gel transition

ii) is capable of maintaining cells in culture for at least 1 hour, 2, 3, 4, 5, l 6, 7, 8, 9, 10, 12, 15, 20 or 24 hours or at least 1, 2, 3, 4, 5, 6, 7, 8, 9, 10, 11, 12, 13, 14, 21, 25, 28, or 31 days, preferably in 3D;

iii) is capable of supporting proliferation of cells, preferably in 3D;

iv) is capable of enabling extraction of viable cells;

v) is capable of supporting transition of cells to a second or further hydrogel;

vi) is capable of maintaining a three dimensional structure;

vii) comprises nanofibers of between 2 and 20 nm in width; and

viii) exhibits stability for at least 1, 2, 3, 4, 5, 6, 7, 8, 9, 10, 11, 12, 13, 14, 21, 25, 28 or 31 days.

ix) is made by a method of the invention.

In a preferred embodiment of the seventh aspect, the hydrogel is prepared by a method of the invention. Preferably, the preferred embodiments of the first to sixth aspects of the invention apply to the seventh aspect, mutatis mutandis.

In an eighth aspect of the invention, there is provide a kit for making a hydrogel of the invention, comprising either dry peptide, peptide solution or peptide gel, preferably a peptide gel, and instructions for carrying out a method of the invention; and optionally one or more of base, buffer, media, cell, a vessel, and means for introducing the gel into a subject. The peptide comprises a portion which is amphiphilic.

### BRIEF DESCRIPTION OF THE DRAWINGS

Embodiments of the invention are further described hereinafter with reference to the accompanying drawings, in which:

FIG. 1 is a diagrammatic representation of octa-peptides having alternate charged/non-charged residues suitable for use in the present invention (Sainini et al 2009, Soft Matter 5 193-202);

FIG. 2 is a diagram showing the assembly of peptides into a hydrogel. The peptide is dissolved in dH₂O, forming a network composed of fibres approximately 4 nm in diameter. Gel stiffness and/or stability can be altered by changing the peptide concentration. Gels can recover structure after disrupting/sheer stress;

FIG. 3 shows the development of embryonic stem cells, and indicates the markers exhibited at each stage;

FIG. 4 shows photographs of gels at concentrations of 17.5, 20 and 25 mg/ml of peptide using the peptide FEFEFKFK and final concentrations of 1×10⁶ cells/ml, at days 1, 3, 6 and 9. Embryonic stem cells seeded as single cells remain viable and all gels tested support the proliferation of embryonic stem cells into large rounded aggregates.

FIG. 5 shows the results of flow cytometry at day 5 for the gels of FIG. 4. The results indicate that increasing the concentration from 17.5 mg/ml to 25 mg/ml of peptide results in an increase from 89.5% of cells being Oct4 positive to 93.8% being Oct4 positive.

FIG. 6 is a graph showing the results of sub-culturing the cells, with an 89.79% positive starting population. The sub-culturing established that the cells can be successfully retrieved from the gels and re-seeded into fresh gels, where they continue to grow in rounded aggregates. The gel stability is not compromised. Monitoring of the markers showed that the Oct4-GFP cells, which were cultured for 15 days and passaged 3 times, maintained their Oct4-GFP levels throughout the culture period.

FIG. 7 is a graphical illustration showing the results of cell density analysis for the 20 mg/ml gels comprising Oct4-GFP cells seeded at 1×10⁶, 5×10⁵ and 1×10⁵ and counted at days 1, 3 and 5. These results show that Oct4-GFP is maintained at all concentrations. A steady increase after one day was observed in 1×10⁵ cells/ml gel. A rapid increase from day 1 to day 3 was observed at 5×10⁵ and 1×10⁶ cells/ml, with the population reaching a plateau after day 3.

FIG. 8 is a graphical illustration showing the results of cell density analysis for the 25 mg/ml gels comprising Oct4-GFP cells seeded at 1×10⁶, 5×10⁵ and 1×10⁵, counted at days 1, 3 and 5. The results show that there was a slight decrease in Oct4-GFP, although levels were still high after 5 days. Continuous proliferation was observed at all concentrations.

FIG. 9 is a graphical illustration of the change in cell density relative to the seeding density for the 20 mg/ml and 25 mg/ml gels. Faster proliferation was observed within 20 mg/ml gels, and fastest proliferation at 1×10⁵ cells at 20 mg/ml gel;

FIG. 10 is a photograph showing the results of cell density analysis;

FIG. 11 is a photograph showing the results of pluripotency analysis, showing that the mouse embryonic stem cells retain pluripotency during culture in the gels;

FIG. 12 shows the results of RNA analysis in agarose gels. Cells trypsinised as described for sub-culture (cells counted; cell/gel debris washed with 5 ml PBS; cells/gel debris spun 1000 rpm for 5 minutes; excess PBS removed; cells/gel debris directly disrupted by RNA lysis buffer and processed for RNA extraction following standard protocol. C=cell pellet 1.2×10⁶ cells; G1=1 300 μl gel=1.5×10⁶ cells; G3=3 300 μl gel=3×10⁶ cells; This figure shows that RNA can be extracted from cells grown in a hydrogel of the invention.

FIG. 13 shows the photographs of gel injection into lamb hearts using PBS as a control;

FIG. 14 is a photograph of three FEFEFKFK hydrogels, set in standard mouse ES media (A) and human ES media mTeSR™ (B) and StemPro™ (C).

FIG. 15 Schematic diagram demonstrating gelation of the hydrogels via self-assembly of functionalised and non-functionalised peptides. The functional peptide can be dosed in by simple mixing and the self-assembly process ensures that the functional peptide is incorporated in the network. The simplicity of the structure formed, cross β-sheet, ensures that the functional group is placed on the surface of the fibre and is therefore available/accessible.

FIG. 16 Typical Fourier Transform IR spectra of hydrogel (here FEFEFKFK) showing a strong band at ˜1615-1630 typical of the peptide adopting a β-sheet conformation. Hydrogel prepared in water.

FIG. 17 Example of amino acid substitution possible without altering the gelation ability of the peptide (gel storage modulus vs peptide concentration). Sequence design and peptide concentration can be used to control the mechanical properties of the hydrogels. Hydrogels prepared in water.

FIG. 18 Typical rheological experiment showing the shear thinning properties of the hydrogels and therefore their injectability. An oscillatory shear is applied for 250 s (shear 1% frequency 1 s⁻¹ peptide VEVEVKVK) and the storage modulus of the gel measured. At 250 s a continuous shear is applied for 5 s to liquefy the gels, storage modulus goes to 0. Subsequently the oscillatory shear is re-establish to investigate the recovery of the mechanical properties of the gel, increase in storage modules. Insert gel injected through a 30 G needle.

FIG. 19 Typical storage moduli of gels prepared in water (pH 3), neutralised with NaOH (pH 7) prepared in cell culture media.

FIG. 20. Phase contrast images of NCL-1 hES cells cultured within 20 mg/ml FEFEFKFK hydrogels at day 3 (a) and day 4 (b). Scale bar: 400 μm

FIG. 21. Confocal images of NCL-1 hES cells cultured within 20 mg/ml FEFEFKFK hydrogels for five days. Cells were assessed for proliferation using EdU incorporation. Higher magnification images to show that hollow cavities from within the cell aggregates reminiscent of EB formation (d,e,f). a+d) phase contrast. b+e) DAPI nuclear stain. c+f) EdU incorporation. Scale bars: a-c 50 μm, d-e 25 μm.

FIG. 22. Live/dead staining of NCL-1 hES cells cultured within 20 mg/ml FEFEFKFK hydrogels for three days. Viable cells were present as indicated by green staining, with some cell death evident (red staining). Scale bars: a) 500 μm, b) 75 μm.

FIG. 23. Live/dead staining of NCL-1 hES cells cultured within 20 mg/ml FEFEFKFK hydrogels for eight days. Viable cells were present as indicated by green staining, with some cell death evident (red staining). Cells were observed to have spontaneously differentiated. Scale bars: a) 500 μm, b) 250 μm

FIG. 24. Live/dead staining of HuES7 hES cells cultured within 20 mg/ml FEFEFKFK hydrogels for three (a) and five (b) days. Viable cells were present as indicated by green staining, with some cell death evident (red staining). Scale bars: 500 μm.

FIG. 25. Live/dead staining of NCL-1 hES cells cultured within 20 mg/ml FEFEFKFK hydrogels for three days. Cells were either seeded as dissociated single cells (a) or dissected colony pieces (b). Viable cells were present as indicated by green staining, with some cell death evident (red staining). Both methods facilitated formation of viable cell aggregates. Scale bars: 500 μm.

FIG. 26. Live/dead staining of MSCs cultured within 20 mg/ml FEFEFKFK hydrogels for one, three and ten days. Cells were seeded as single cells. Viable cells were present as indicated by green staining, with some cell death evident (red staining). A shift in morphology to a more spread phenotype was evident at day ten. Scale bars: 250 μm.

FIG. 27. Cell viability assay using live/dead staining shows that HUCPVCs were successfully extracted from a 20 mg/ml gel and cultured into a fresh 20 mg/ml gel. At day 1 there is initial cell death as evidenced by the red cells; however this decreases at days 3 and 5, where there is a higher proportion of live cells, shown in green

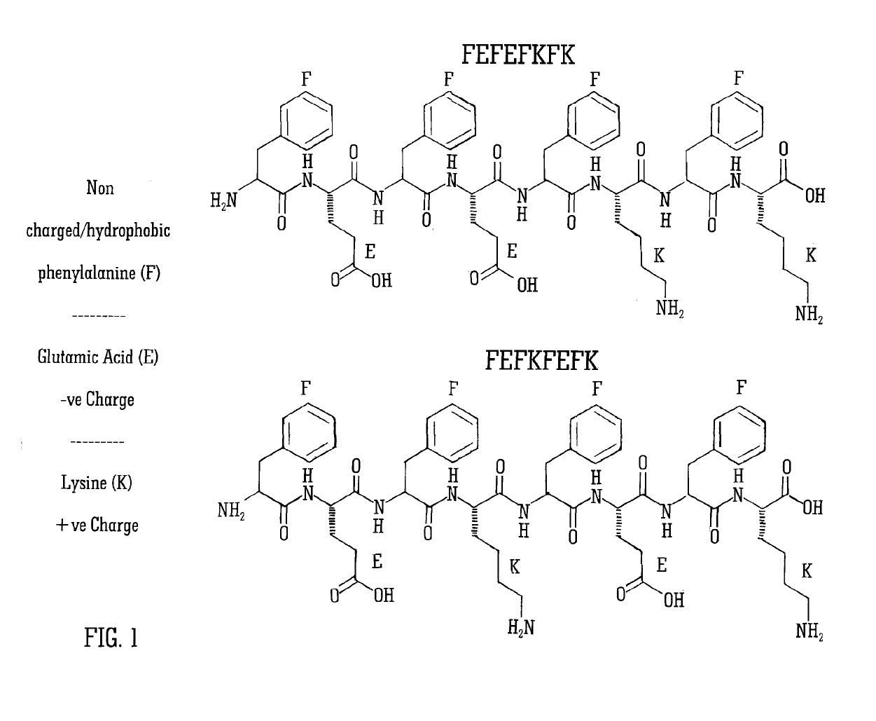

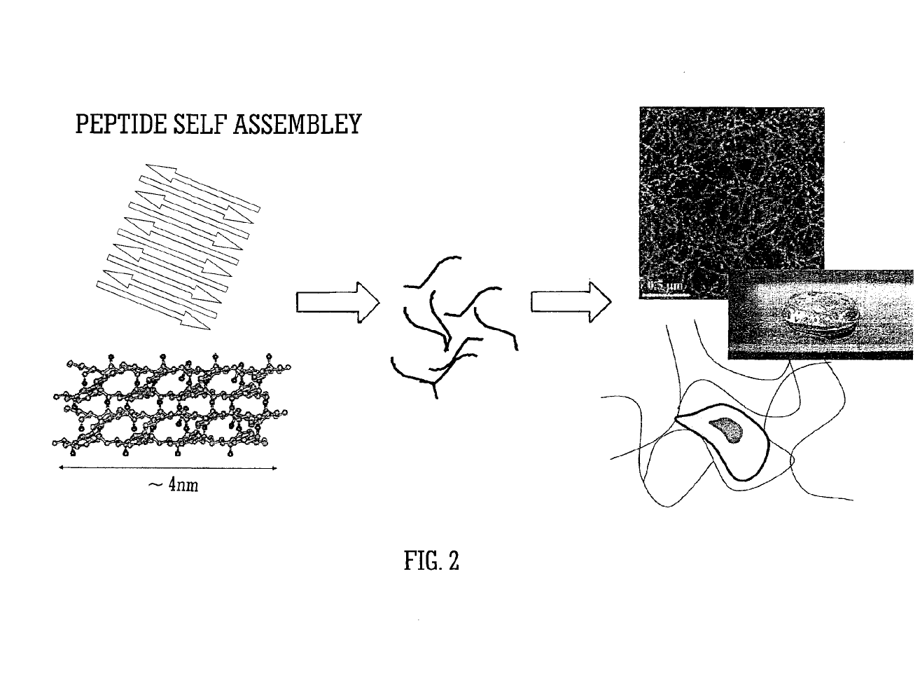

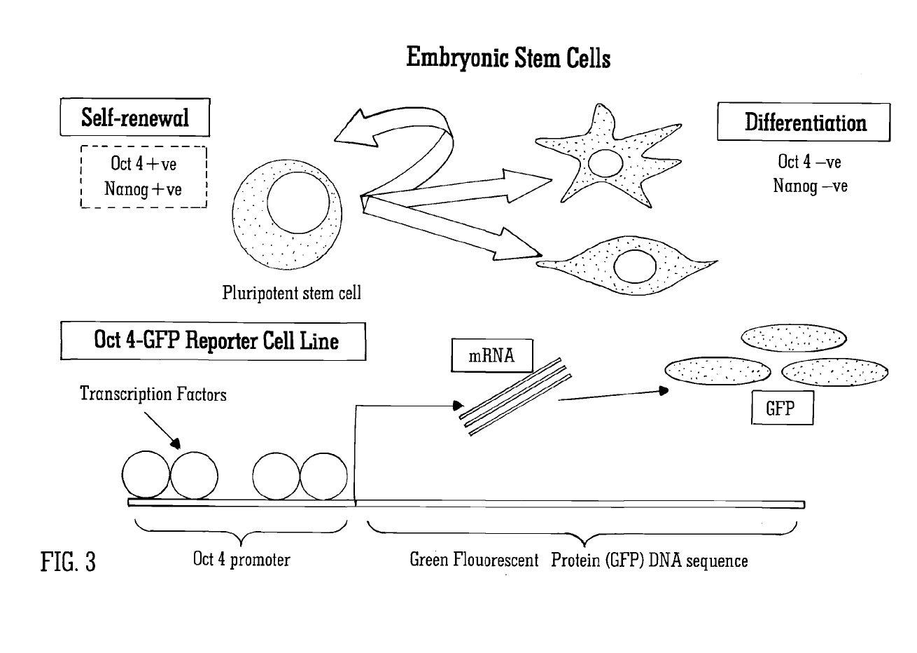

### DETAILED DESCRIPTION

The present invention relates to a novel protocol for making a hydrogel, and is based upon the discovery by the present inventors that the risk of loss of cells can be reduced and the reliability of the method of making the hydrogel can be improved by adopting a two stage process in which a hydrogel is formed, liquefied, and then reformed with encapsulated cells. This method has the clear advantage that the cells are added to a solution which is known to be capable of forming a hydrogel, in contrast to the prior art in which cells are added to a peptide solution which has not previously formed a hydrogel, and so there remains a significant risk of cell sacrifice at this stage. The variability between batches of peptides, and the degree of alteration of a characteristic required to trigger the formation of a hydrogel for any particular peptide batch has meant that this remains a significant problem in the art, and optimisation procedures have not been available. The present inventors have identified a marker of the ability of a peptide solution to form a hydrogel. They have observed that optical transparency of a peptide solution after addition of a base thereto is indicative of the ability of that solution to form a hydrogel. Thus, the method of the present invention enables for the first time a user to reliably predict whether a particular peptide solution is capable of forming a hydrogel, before cells are added, thus significantly reducing the risk of cell sacrifice. The optimisation process provided by the present invention further enables the preparation of hydrogels using any peptide batch, such that gel to gel passage becomes a realistic option.

A hydrogel of the invention will preferably be non-cytotoxic, although it is envisaged that there may be embodiments of the present invention where cytotoxic hydrogels may be tolerated. It may be capable of supporting a variety of all types, including prokaryotic and eukaryotic cells. Examples include insect cells, yeast, bacteria, plant cells, algae, and animal cells, preferably mammalian cells. A hydrogel of the invention is mechanically self-supporting. It is composed of peptides, assembled into β-sheets which form nanofibres.

In the present invention, a hydrogel is a gel comprising water. Thus, water is the medium in which the structural components of the hydrogel become dispersed and undergo self-assembly to form a scaffold. The structural components of the hydrogel of the invention may be any suitable molecules, either naturally occurring or synthetic, but are preferably peptides comprising an amphiphilic portion, which upon contact with water and under suitable conditions will self-assemble to form nanofibres which are the basis of the scaffold. By “self-assembly” is meant the spontaneous organisation of the peptides into well-ordered structures via reversible, non-covalent interactions such as hydrogen bonds, disulphide bonds, Van der Waals interaction, electrostatic interactions, hydrophobic interactions and π-π stacking.

“Gelation” is a term used to describe the effect of self-assembly of the peptides in the liquid, which causes a transition from a liquid state to a gel-like state. The formation of a three dimensional network introduces features of increased stiffness, viscosity, elasticity and reduced pourability, and consequently is termed a “gel”. The term “gelation” may be used interchangeably with terms such as “setting”. The characteristics of a gel are that it is jelly-like, being a liquid dispersed among a solid scaffold. Preferably, a gel of the present invention will be incapable of flow. Thus, an increase in flow indicates a decrease in stability of the gel, as it reverts to a liquid state. The hardness of the gel may vary, depending upon its purpose, and will be a factor of the peptide concentration and any functional groups present. Preferably, a hydrogel of the invention will have a degree of flexibility similar to living tissue. The degree of flexibility may be termed the visco-elastic properties of the gel, measured by oscillatory rheology or any other suitable methods.

A “scaffold” is a three dimensional structure which is capable of supporting cells. The scaffold is the assembly of peptides in the gel. The cells may be encapsulated by the scaffold, meaning that they are evenly or unevenly dispersed throughout the three dimensional structure. Alternatively, cells may be provided on the surface of a scaffold. A hydrogel scaffold of the invention may typically have a β-sheet secondary structure, such that it will provide an absorbance of between 218 nm and 195 nm, using standard circular dichroism (CD) techniques. FTIR can also be used to determine the presence of β-sheets, the spectra will present a strong absorption band at 1620-1645 cm-1 in water and 1615-1640 cm-1 in deuterated water.

A hydrogel of the invention will preferably comprise at least 80% (w/w) water, more preferably at least 85%, more preferably at least 90%, and most preferably at least 95, 96, 97, 98 or 99% water (w/w). The advantage of a hydrogel having a high water content (i.e. 80% or above) is that it allows cells encapsulated in the hydrogel to migrate within the hydrogel, and therefore communicate and/or make intercellular contact. In addition, a water content of at least 80% allows small molecules or other non-cellular material to diffuse within the hydrogel and become available to cells. In this manner, the maintenance, growth, differentiation and modification of the cells can be controlled by the addition to the hydrogel of external factors. It is envisaged, however, that a hydrogel of the invention may in certain embodiments have a lower water content, for example at least 50%, 60%, or 70% (w/w).

A hydrogel formed according to the invention may have varying amounts of solid material, which may be expressed as a percentage by weight of peptides, ranging from 0.1% to 10% (more preferably from 1.5% to 4%, more preferably from 2% to 3%.

A hydrogel may be charged or uncharged, independently of the charge of any particular amino acid or peptide present in the hydrogel.

A hydrogel made by a method of the invention may be referred to herein as a hydrogel of the invention.

A hydrogel produced according the invention may have a desired degree of stiffness. This may be measured using techniques known and available to persons skilled in the art, and may range from 1 Pascal (PA) to about 100 000 Pascal, or more preferably from about 1 to 50 000 Pascal, 1 to 25 000 Pascal, 1 to 10 000 Pascal, 1 to 5 000 Pascal, 1 to 2000 Pascal, 1 to 1000 Pascal, 1 to 500 Pascal, 1 to 250 Pascal, 1 to 100 Pascal, or between 10 to 100 Pascal.

By “stable” is meant that the hydrogel substantially retains its structural and/or chemical integrity, and does not substantially decompose or disintegrate within a specified period. A stable hydrogel will preferably retain its nanofibre scaffold for the duration of this period. A specified period may be a period of proposed use of the gel, for example the period of cell culture. A specified period during which a hydrogel of the invention retains at least 70% of stability is preferably at least 1, 2, 3, 4, 5, 6, 7, 8, 9, 10, 11, 12, 13, 14, 21, 25, 28 or 31 days. Preferably, a stable hydrogel also retains its ability to support cell culture. By “substantially” within this context is meant that it preferably retains at least 70%, 75%, 80%, 85%, 90%, 95%, 96%, 97%, 98% or at least 99% of its stability, relative to its stability on day 1. The period of stability is dependent upon various factors, including for example peptide length and sequence, such as gel concentration, number of cells encapsulated and the type of cells.

Gel stability may also be assessed by determining the degree of gel degradation in the presence of cells, for example as a factor of gel volume. Therefore, as a gel loses stability, it loses volume. Thus in the present invention, a stable hydrogel will preferably undergo less than 50%, preferably less than 30% loss of gel volume by erosion, over the specified period. A hydrogel of the invention may lose 50%, or preferably less than 30% of its volume by the end of day 1, day 2, day 3, day 4, day 5, day 6, day 7, day 8, day 9, day 10, day 11, day 12, day 13, day 14, day 21, day 25, day 28 or day 31. The present inventors have found that the period of stability of a hydrogel will depend upon factors such as the peptide concentration used, the cell type, and cell number supported by the hydrogel. For instance embryonic stem cells seeded in 20 mg/ml gels last for 8 days, whilst embryonic stem cells seeded in 25 mg/ml gels last for 14 days, and gels supporting slower growing cells have shown stability for longer than 30 days (ie. less than 30% loss in volume over this period).

The volume of a hydrogel is generally measured by eye, simply by estimating the approximate loss in volume over the specified period. Other techniques may also be used. As the surface of the gel is washed, the gel may erode and parts of it may be washed away. The remainder of the gel maintains the properties of the original gel, but is simply reduced in volume. In an embodiment, this effect can be reduced by careful use of cell culture transwell inserts that allow media to be refreshed with minimal gel disturbance. An unstable gel is one which has lost at least 30%, 40% or at least 50% of its volume by the end of the specified period. An unstable gel may be capable of supporting cell culture, but does not provide optimal cell culture conditions.

Stability of a hydrogel may be also assessed using any known method available in the art. Suitable methods for assessing the stability of a hydrogel include analysis of gel strength, which will decrease with loss of stability. Gel strength may be determined using, for example, a compression test, where the degree to which a gel can withstand compression of a particular value will decrease with loss in strength, thus indicating loss in stability. Optimal gel strength for a hydrogel of the present invention is between 1 and 50 kPa. Lower strength gels may be preferred, in that they allow cell migration and cell-cell communication within the gel. The strength of the gel (which affects volume and stiffness, and therefore features such as cell migration and extraction) can be governed by peptide concentration, hydrophobicity, size and sequence. Peptides may be cross linked to increase gel strength.

Preferably, a hydrogel produced by the method of the invention is stable in any suitable aqueous media, including for example cell culture media (DMEM (Dulbecco's Modified Eagle Medium), KO-DMEM (Knock Out DMEM), Iscove's Modified Dulbecco's Media (IMDM), StemPro™, mTeSR™, water, saline (for example phosphate buffered saline, Earle's balanced salt solution and Hank's balanced salt solution), and serum. Preferably, the media will have an osmolarity which is compatible with any cells of the gel. A hydrogel made by the method of the invention may be stable in non-aqueous conditions such as in tissue. A hydrogel made by a method of the invention is preferably stable in vivo or in vitro. As such, a hydrogel of the invention may be maintained, stored or transferred in any of these conditions.

A hydrogel of the invention is preferably stable under biological conditions. Biological conditions may be defined as an ionic strength of between 0.01M to 1.0M, preferably 0.05M to 0.5M, most preferably 0.1M to 0.2M. The ionic strength will vary depending upon the whether the biological conditions are in vivo or in vitro. Ionic strength for in vivo applications may be between 0.1M and 1.0M. Ionic strength for in vitro applications may also be between 0.1 and 1.0M. Biological conditions in terms of pH include a range of between 6 and 9.5. The biological conditions in terms of pH will vary depending upon the condition to be treated. For example, it is known that chronic wounds may have a pH of between 6 and 8. Typically, however, it is preferred that the hydrogel is stable at neutral pH, so between 6 and 8, preferably between 6.5 and 7.5, more preferably between 6.7 and 7.7, more preferably between 6.8 and 7.3, more preferably 6.1 and 7.1, more preferably pH 7. Biological conditions in terms of temperature include a range of between 24° C. and 38° C. Preferably, biologically acceptable temperature conditions range between 32° C. and 38° C., more preferably between 34° C. and 37° C., most preferably at 37° C. Biological conditions typically refer to atmospheric pressure.

Characterisation of a hydrogel prepared by the invention, may be determined using any technique known in the art, for example, Circular Dichroism (CD), dynamic light scattering, fourier transform infrared, atomic force microscopy small angle x ray scattering, and scanning or transmission electron microscopy. Such techniques can provide information regarding the pore size, β-sheet formation, fiber diameter and length, elasticity, and the degree of secondary structure formation. Mechanical testing may be used to assess volume, strength, elasticity, swelling and hydration.

A hydrogel of the present invention may have a peptide concentration at or above a Critical Gelation Concentration, which is between 15 and 40 mg/ml, preferably between 17 and 30 mg/ml, more preferably between 20 and 25 mg/ml. This concentration refers to the amount of peptide per ml of final volume of the hydrogel, and may be calculated as described herein.

In the present invention, a peptide is a string of amino acids which are connected by peptide bonds. Amino acids can be natural, non-natural, or a combination thereof. They may be L-amino acids or D-amino acids. L-amino acids may be preferred where the hydrogel is to be introduced into a subject. Peptides may be chemically synthesised or purified from natural or recombinant sources.

The peptides of the present invention comprise an amphiphilic portion, meaning that this portion of peptide is composed of alternating hydrophobic and hydrophilic amino acid residues. The alternating pattern may be one hydrophobic—one hydrophilic residue, or may be alternating two hydrophobic, two hydrophilic residues, etc. Other suitable patterns will be known to persons skilled in the art. The former is preferred. Pairing of complementary amino acids between peptides enables the formation of higher order structures, such as β-sheets. Complementary amino acids are those which are capable of forming hydrogen bonds or ionic interactions.

Intrapeptide distance is the spacing between peptides, and in a stable hydrogel will preferably be between 0.5 and 5 angstroms, more preferably 1, 2, 3 or 4 angstroms. Typically, the amphiphilic portion of peptides will self-assemble into anti-parallel β-sheets which then form higher order structures such as nanofibres. It is recognised that the greater the number of bonds which form between hydrophilic residues in neighbouring peptides, the greater the stability or strength of the resulting gel. Thus, the stability of a hydrogel may be influenced by adjusting the amino acid sequence of the peptides, or the populations of different peptides present. By varying the charge within the peptide, the folding rate can be varied, by varying the lateral and facial intermolecular hydrophobic interactions and/or the intramolecular hydrophobic reactions, the folding self assembly of the peptides as well as the material properties of the hydrogel can be varied; varying the intramolecular and/or intermolecular bond formation can be used to vary the folding, self assembly and final properties of the gel; and finally any turn regions of peptides can be designed to further control folding and self assembly. It is also envisaged that the hydrophobic residues may interact with each other and exclude water, thus adding to the stability of the nanofibres. In an embodiment, the β-sheets formed by neighbouring peptides will preferably be spaced at about 0.5 nm apart. If the peptide is present at or above the Critical Gelation Concentration, the nanofibres form a network (or mesh) of fibres, which acts as a scaffold for the cells. The peptide concentration used in the first step of the method of the invention dictates the amount of peptide present in the hydrogel, and therefore whether or not a critical concentration of nanofibres may be achieved to enable the formation of a hydrogel. Preferably, the nanofibres are less than 100 nm, preferably less than 50 nm, preferably less than 20 nm, preferably between more preferably between 1 and 10 nm thick, more preferably between 3 and 6 nm, more preferably between 4 and 6 nm. It is envisaged that there may be variance in the thickness of the nanofibres within any single hydrogel. Preferably, substantially all the nanofibres fall within the desired range. The nanofibres may be of any length and may form a continuous network. The thickness and length of the nanofibres in a hydrogel may be determined by any suitable method, as described therein.

Peptides used in invention are typically provided as salts, and therefore may comprise a counter ion which is washed out during washing of the gels. Preferably, the peptides are free of impurities, for example biological material from the synthesis or purification process.

Preferably a hydrogel of the invention will comprise at least 50%, 60%, 70%, 80%, 90% or 95% or 99% peptides having the same sequence.

For the present invention, the amphiphilic portion of a peptide is preferably between 2 and 100 amino acids, more preferably between 2 and 50 amino acids, more preferably between 2 and 20 amino acids, more preferably between 4 and 18 amino acids, more preferably between 6 and 16 amino acids, more preferably between 8 to 14, more preferably between 8 and 12, more preferably 8 and 10, and most preferably 8 amino acids.

The amphiphilic portion of a peptide causes gelation, by its ability to form a higher order structure by self assembly, as described herein. This portion may be referred to as the gelator portion. A peptide may comprise a non-amphiphilic portion, of any suitable length depending upon its function. Typically, so as not to adversely affect the assembly of amphiphilic portions of peptides into beta sheets and nanofibres, a non-amphiphilic portion (also referred to as a second portion, or functionalised portion or domain) may be less than 50 amino acids, preferably less than 30 amino acids in length, and may be less than 20 amino acids, or 10 amino acids. Thus, it is envisaged that a peptide for use in making a hydrogel according to the invention may comprise one or more first amphiphilic portions, for assembly into higher order structures, and optionally one or more non-amphiphilic second portions, which are not required for assembly of the peptide into a higher order structure but may have an alternate function. A second portion may be provided between two amphiphilic first portions, or at one or both ends of an amphiphilic first portion. This may be the N-terminus and/or the C-terminus of the first portion. A second portion may be provided between two amphiphilic portions and an additional second portion may be provided at an end of an amphiphilic portion, such that at least one second portion in sandwiched between two amphiphilic portions.

Examples of second portions include sequences for functionalising the peptide, for example binding sites for target moieties such as proteins, small molecules or antibodies, chemo-attractants, ligands, small molecules, agonists and antagonists, carbohydrates or cells, or for cross linking peptides or for attracting markers such as fluorescent or radio labels. An example of a preferred functional site is a glycosaminoglycan binding site, thus enabling the peptides within the gel to bind glycosaminoglycans such as heparin which increases growth factor stability and retention in the gel and may alter the physical properties of the gel. Alternatively, functional sites may be introduced into the amphiphilic portion, for example by altering a side chain. Thus, the peptides can be tailored to have specific functions or binding capabilities, and indeed a key benefit of the gels of the present invention is the ease with which functional groups (for example short peptides which bind glycosaminoglycans or motifs from extracellular matrix proteins to increase cell attachment) can be introduced.

A non-amphiphilic portion may be covalently linked to an agent, compound or entity which affects a biological or biochemical event. For example, biological agents include proteins, amino acids, peptides, nucleic acids, carbohydrates, sugars, lipids, nucleoproteins, glycoproteins, lipoproteins, steroids, growth factors, chemoattractants, drugs and therapeutics.

The provision of a functional group within a peptide used to make a hydrogel of the invention may affect the stability of the hydrogel. Certain preferred functional groups will increase the stability of the hydrogel. Compensation in terms of volume and concentration of the peptide for the presence of any functional groups is preferably made at the outset, when calculating the amounts of reagents and cells required.

In embodiments of the invention, it may be preferable to consider the peptides in terms of size rather than length of sequence. Preferred amphiphilic peptides for use in the invention may range from 150 Da to 5 kDa.

Herein, reference to “peptide” includes reference to a single peptide or to a batch of peptides.

The amino acids used in the peptides of the invention may be natural or non-natural (i.e. artificial) amino acids. Derivatives or analogues of the amino acids may be used. The carboxy and/or amino termini of a peptide may be protected or may not be protected.

Hydrophobic amino acid residues include alanine, cysteine, isoleucine, leucine, methionine, phenylalanine, proline, tryptophan, valine or tyrosine. Hydrophilic amino acid residues are arginine, asparagine, aspartate, glutamine, glutamate, histidine, lysine, serine and threonine. It may be possible to change a hydrophilic residue for another hydrophilic residue, or a hydrophobic residue for other hydrophobic residue with little effect on structure or function of the gel. In the present invention, peptides comprising any alternating hydrophobic and hydrophilic residues may be used, or alternatively pairs of hydrophilic and hydrophobic residues, or indeed other suitable patterns which allow the peptides to self-assemble into a scaffold, to form a gel. Preferably, however, the amphiphilic portion of the peptides preferably comprise phenylalanine, glutamic acid and lysine. Preferably, the amphiphilic portion of the peptides comprise alternating phenylalanine and glutamic acid residues, and/or alternating phenylalanine and lysine residues. Suitable substitutions include valine and leucine. Preferably, the amphiphilic portion of the peptide comprises the sequence FEFE and/or FKFK. More preferably, the amphiphilic portion of the peptide comprises the sequence FEFEFKFK.

To maintain ability of peptides to self assemble into a higher order structure, certain amino acid changes may be made without affecting this ability. For example, lysines and arginines are interchangeable, as are glutamates and aspartates. Phenylalanine is interchangeable with leucine, tyrosine and valine.

A batch of peptides may be homogenous (i.e. composed of peptides identical in nature) or heterogeneous (i.e. composed of two or more different peptides). They may be identical or different in terms of size and/or sequence and/or composition and/or structure (for example, the presence of one or more functional groups). Preferably, a homogenous batch of peptide includes peptides of the same length and the same sequence. In a heterogeneous batch, the peptides must be structurally compatible, meaning that they are able to self-assemble with each other to form nanofibres. They may be of different lengths, although it is preferred that they are of identical lengths, but different in sequence. Where different in length, it is preferred that they include peptides where the amphiphilic portion of some peptides are twice the length of the amphiphilic portion of other peptides.

Peptide for use in the present invention may be provided in dry form, which includes either dehydrated or lyophilised forms. Typically, a peptide will be provided in lyophilised form. Alternatively, a peptide may be provided as an aqueous solution, for example pre-dissolved in water at a predetermined concentration and volume. For the methods of the invention, a dry form is preferred. In a preferred embodiment of the first aspect, the peptide where provided in dry form is used to produce a peptide solution. Preferably, this is achieved by dissolving or reconstituting the peptide in an aqueous or non-aqueous solvent. Preferably, this is conducted at between 60 and 95° C., preferably between 60 and 65° C., 65° C. and 70° C., 70° C. and 75° C., 75° C. and 80° C., 80° C. and 85° C., 85° C. and 90° C. or between 90° C. and 95° C. The time taken to produce the solution may be at least 15, 20, 25, 30, 35, 40, 45, 50, 55, 60, 90, 20, 150, 180, 210 or 240 minutes.

Examples of suitable solvents include any of those which are suitable for use with cells. A preferred solvent is water. The amount of solvent used is preferably the minimum required to dissolve the peptide. The concentration of the final peptide solution used to produce the first solution will preferably be between 30 and 50 mg/ml, more preferably 35 and 45 mg/ml, more preferably between 38 and 42 mg/ml, more preferably 25 mg/ml.

The hydrogel prepared by step 1) or step 5) of the method of the first aspect of the present may be frozen, either with or without cells. A frozen hydrogel may be defrosted and used as described herein. A hydrogel prepared by the method of step 1) of the first aspect may be stored, preferably at about 4° C., for periods of time up to 12 months.

Thus, there is provided a method of encapsulating cells in a hydrogel, comprising:—
5) providing a pre-formed hydrogel;
6) liquefying the hydrogel;
7) combining the liquefied hydrogel of 6) with i) cells and ii) media to form a second liquid;
8) maintaining the second liquid of 7) under conditions suitable for the formation of a hydrogel.

In an embodiment, the pre-formed hydrogel is prepared by steps 1) to 3) of the first aspect.

The pre-formed hydrogel may have been previously frozen and defrosted, and/or stored at about 4° C. It may be liquefied by mechanical disruption of the three dimensional scaffold, for example as described herein, or chemically.

In an embodiment, the method comprises removing a pre-formed hydrogel from storage, and defrosting if necessary by maintaining at room temperature for a suitable period of time.

In the context of the present invention, optically transparent means that a non-visually impaired person can see through the liquid or hydrogel sufficiently well to read black text of font size 12 or lower on the other side of the gel. An optically transparent liquid or hydrogel may comprise aggregates, but which do not significantly obscure the transparency of the liquid or gel. Suitable methods may be used to determine optical transparency, such as UV-Visible spectroscopy. The degree of transparency is expressed as percentage transmittance, wherein increased transmittance is associated with increased gel clarity, and therefore increased stability and reproducibility. Preferably, the values are expressed as percentage transmittance of a specified wavelength of light at 650-750 nm. Values indicative of an optically transparent liquid or gel are greater than 30%, 40%, 50%, 60%, 70%, 80% or 90% transmittance of light between a wavelength of 650-750 nm. Preferably the first liquid has a transmittance greater than 30% of light of a wavelength between 650-750 nm. Preferably, the second liquid has a transmittance greater than 35% of light of a wavelength between 650 and 750 nm.

A hydrogel of the present invention may preferably have a final volume and/or shape which is dictated by the application of the hydrogel. The size and shape may be pre-determined by casting the gel in a pre-formed mould. In embodiment, a gel may be cast (i.e. printed) as a microarray. This may comprise two or more layers of gel. The desired final volume of the gel will be dictated by its proposed application and the number of cells to be encapsulated or cultured thereon.

The three dimensional structure means that the hydrogel is capable of encapsulating cells meaning that substantially all the cells are completely enclosed by the hydrogel, as opposed to only remaining on the surface thereof. It is envisaged, however, that for certain applications it may be preferable for the hydrogel to be in two-dimensional form, where the cells are only partially encapsulated, or lie on a surface of the gel.

Cell density in the hydrogel may be 10⁵/ml, between 2×10⁴ and 1×10⁷/ml, preferably between 5×10⁵ and 5×10⁵/ml. Culture conditions within the gel are preferably as close to physiological conditions as possible, for example pH 6-8, more preferably pH 7-7, preferably pH7.4. Physiological temperatures range between 30° C.-40° C. Preferred temperature, in particular for mammalian cells, is between 32° C. and 38° C., more preferably between 35° C. and 37° C.

A hydrogel of the invention, when subjected to sonicating, vortexing, tapping, or physical agitation, such as shaking or pipetting or stirring, will not retain the physical properties such as stability, elasticity and viscosity, which it had prior to agitation. Thus, the hydrogel of the invention may be wholly or partially liquefied upon mechanical or physical agitation. Alternative methods include chemical liquefaction, for example by addition of an enzyme. Cell compatible methods are preferred where it is desirable to maintain viability of the cells.

The amount of peptide used in step 1) of the method of the invention may be calculated as follows:

i) determining the desired final volume of the gel;

ii) determining the desired final concentration of the gel;

iii) calculating the amount of peptide to be used as volume (ml)×final concentration (mg/ml).

The above calculation is based upon the use of dry peptide. A person skilled in the art will be able to adjust the calculation where a peptide is provided as a solution.

The mixing with base in step 1) of the method ensures that any variability in the pH of a peptide batch is removed, and that reproducibility of the gel can be achieved. The base used in the method of the invention for mixing with the peptide may be any alkali, preferably of pH 8 to 14, more preferably 13. The concentration of the base used in the invention for mixing with the peptide may range between 0.5-2.5M, preferably 0.5M. Preferred bases for use in the invention include sodium hydroxide, potassium hydroxide, calcium hydroxide, magnesium hydroxide, disodium phosphate, monopotassium phosphate, monosodium phosphate, and sodium bicarbonate. The base may be in dry form, for example as a powder, or may be provided as a solution. For accuracy, the latter is preferred.

The amount of base to be added to the peptide is calculated through an optimisation method. This method comprises the steps of:
i. determining the final volume of the gel to be made;
ii. determining the final concentration of the gel;
iii. calculating the amount of peptide to be used as volume (m/I) and final concentration;
iv. mixing the peptide of ii) or iii) with the base to produce a first solution;
v. determining the optical transparency of the first solution;
vi. if the first solution is not optically transparent, continuing to add base until an optically transparent solution is obtained;
vii. maintaining the solution under conditions suitable for formation of a hydrogel;
viii. assessing gel stability; wherein if unstable, discarding and repeating steps i) to vi) wherein a reduced amount of base is added in step iv);
ix. calculating the total amount of base added per mg of peptide used to obtain an optically transparent first solution and a stable hydrogel.

Optionally, the following steps may be followed:
i. where the gel is not optically transparent or is not stable, increasing the amount of base in step iii) by between 0.3 and 10% of the final gel volume until an optically transparent solution is obtained;
ii. where the gel is optically transparent but unstable, reducing the amount of base in step iii) until the gel is stable.

In the method of the first aspect, the amount of base calculated in step vii) of the optimisation method is mixed with the peptide. Preferably it is added to the peptide, preferably in a step-wise fashion. Preferably, the base is added to the peptide such that the amount of base being added can be carefully controlled. However, it may be appropriate to add peptide to the base, and this is included within the scope of the present invention. Preferably, the first step may comprise maintaining the first solution at between 60° C. and 95° C. until the peptide and the base have formed a homogenous solution.

A cloudy (non-optically transparent) first solution is indicative of insufficient base having been mixed with the peptide. The amount of base may be increased to increase the optical transparency of the first liquid

Preferably, the first solution may be mixed with a buffer solution prior to step b) with media. The purpose of the buffer solution is to ensure a physiologically relevant salt balance is present, minimising osmotic stress on the cells upon encapsulation. Suitable buffer solutions are known to persons skilled in the art, and include for example PBS, Hank's balanced salt solution (HBSS) and Earle's balanced salt solution. Preferably, the buffer is any physiological salt buffer, for example, PBS. Preferably, the buffer is added at a ratio of 1 part buffer:9 parts first solution, as a final dilution. However, it is envisaged that other ratios may work equally well and so the present invention allows for dilutions of 1 part buffer to 8, 7, 6, 5 4, 3 or 2 parts first solution. Preferably, the buffer is added at a ratio of 1 part buffer to 9 parts first solution.

Preferably, the first solution is sterilised prior to addition of the media to produce the second liquid. A preferred method of sterilisation is radiation sterilisation, for example UV sterilisation. Other methods will be known to persons skilled in the art.

Preferably, the amount of media to be added is to provide a media:first solution ratio of between 1:1 and 1:10, preferably 1:2 to 1:5, preferably 1:4. In certain embodiments, the amount of media may be calculated as at least 150, 160, 170, 180, 190 200, 210, 220, 230, 240, 250, 260, 270, 280, 290, 300, 310, 320, 330, 340 or at least 350 μl media per 0.75, 0.8, 0.85, 0.9, 0.95, 1, 1.05, 1.1, 1.15 or 1.2 ml of first solution. These amounts are based upon a peptide concentration in the final gel of between 15 and 50 mg/ml, more preferably 20 mg/ml. it is envisaged that the present invention is suitable for large scale preparation, or for very small volumes, for example in the order of 100 ml, or even smaller, for example in microarrays. Using the rations defined above, a skilled person can calculate the appropriate volumes of solvents, buffers, media etc, and peptide, to produce a hydrogel according to the invention, using the methodology described herein.

The media used in the present invention may be DMEM (Dulbecco's Modified Eagle Medium), KO-DMEM (Knock Out DMEM), Iscove's Modified Dulbecco's Media (IMDM), StemPro™ and mTeSR™, water, saline or serum. Other media known to persons skilled in the art may be used.

Preferably, mixing of the liquefied gel and the media may be addition of the media to the liquefied gel or addition of the liquefied gel to the media. To better control the methodology, addition of the media to the liquefied gel is preferred. Preferably, such addition is step-wise. Preferably, the media and liquefied gel are mixed, for example by pipetting. Alternatively, the media may be placed on top of the liquefied gel, for example by pipetting. The media and the liquefied gel may then be stirred or pipetted to allow mixing. Optionally, the resulting second mixture may be spun to remove any excess bubbles, for example for 2 minutes at 1500 rpm.

The optically transparent second solution is preferably homogenous in coloration, the colour dictated by the media chosen. A homogenous coloration indicates adequate mixing of the second solution.

The second solution may then be maintained under conditions suitable to allow it to set into a gel.

To set the gel, the second solution is poured into a suitable receptacle, for example a well, plate array, or any receptacle shaped according to the final use of the hydrogel. Preferably, a multi-well plate with transwell inserts is used, more preferably a 12-well plate with transwell inserts is used.

The conditions under which the second solution is maintained to allow setting to occur are i) a temperature range of 35° C. to 38° C., preferably 37° C. This temperature may be referred to as the setting or gelation temperature; ii) a time of 5 to 40 minutes, preferably 10 to 30 minutes, most preferably 10 to 20 minutes depending upon its size and concentration.

Preferably, the hydrogel encapsulates cells, which may be maintained therein for culture. By “culture” is meant conditions which allow maintenance, growth, proliferation, and/or differentiation. Thus, the present invention also provides a method of encapsulating cells in a hydrogel. Preferably, the method comprises the method of making a hydrogel as described herein, where the cells are mixed with media, prior to the step of mixing media and the first solution.

The three dimensional structure means that the hydrogel is capable of encapsulating cells, meaning that substantially all the cells are completely enclosed by the hydrogel, as opposed to only remaining on the surface thereof. It is envisaged, however, that for certain applications it may be preferable for the hydrogel to be in two-dimensional form, where the cells are only partially encapsulated, or lie on a surface of the gel.

The amount of cells to be mixed with the media will depend on the desired final concentration of cells in the hydrogel, which can be calculated by determining the final volume of the hydrogel in light of the desired cell concentration. Preferably, the cells are provided in media added to form the second liquid. Cells are suspended in media, and mixed into the first liquid to form the second liquid. Preferably, this takes place in a sterile environment (such as in a class II tissue culture hood), preferably at room temperature.

Any cells may be provided in the media, for culture within a hydrogel of the invention. These may be somatic cells, germ cells, or stem cells. The cells may be eukaryotic, such as human, insect or animal cells; or maybe yeast, plant, algae, or prokaryotic cells. The cells may be cell culture derived, or from tissue homogenate. The cells may be adult or embryonic (including fetal). The invention is particularly suitable for tissue regeneration methods, and for the culture of stem cells.

Stem cells are cells which are undifferentiated, but which have the capacity to generate one or more specialised cell types. Stem cells may be derived from mammalian or non-mammalian sources, and may be somatic or embryonic. Induced pluripotent cells may also be used. Somatic stem cells are found in adult tissue, and may be referred to as adult stem cells. Examples of stem cells include embryonic stem cells, and adult stem cells.

For the purposes of the present invention, any particular cell type includes within its scope progeny thereof, which may be differentiated compared to the parent cell. Thus, for stem cells, progeny thereof may have become fully or partially committed to a particular lineage during culture in the hydrogel. These may include progenitor cells, pluripotent cells, or fully or partially differentiated cells.

Cells may be derived from any tissue type, including for example brain, nervous tissue, bladder, oesophagus, fallopian tube, heart, intestines, gall bladder, pancreas, liver, kidney, lung, ovaries, prostrate, spinal cord, spleen, stomach, testes, thymus, thyroid, trachea, ureter, urethra and uterus.

In an embodiment of the invention, the cells may have been removed from a subject, for culturing in vitro, or for encapsulation in a hydrogel to be used as a vehicle for reintroduction of a cell population into a subject. In an alternative embodiment, the cells may be derived from a source other than the subject into which they are to be introduced. Thus, they may be derived from a different subject, or may be derived from culture.

A hydrogel may be sterilised as a gel, or a first solution or peptide solution. Any standard technique may be used, preferably that which is compatible with cell encapsulation, for example radiation, for example UV radiation.

When referring to a particular cell type, this is the cell type on day 1 of the cell culture period. It is appreciated that during cell culture, the cell type may change, through differentiation for example.

Where the hydrogel encapsulates cells, the hydrogel is preferably maintained for a suitable period of time to culture the cells therein. Within the context of the present invention, the term “culture” may include maintaining the cells (i.e. without significant differentiation, growth or proliferation), but may also include growth of the cells, expansion (i.e. proliferation) of the cells, and differentiation of the cells. By growth is meant in an increase in cytoplasmic and organelle volume (G1 phase), as well as increase in genetic material before replication (G2 phase). By proliferation is meant dividing of a mother cell to form two daughter cells, and typically an increase in the size of the cell population. By differentiation is meant that the cells may adopt a more mature phenotype than their previous phenotype. Undifferentiated and differentiated are relative terms, used in relation to the previous phenotype of the cell to which the terms are referring. With regard to stem cells, undifferentiated may be used to mean that the cells are self renewing stem cells. Changes in phenotype through differentiation can be observed through, for example, changes in morphology of the cells (such as shape), markers, the production and nature of gene products.

The viability, growth and/or differentiation of the cells may also be assessed by monitoring the effects of the cells on the gel, for example the amount of extracellular matrix laid down, which can be measured by any suitable technique, including, for example, oscillatory rheology. They can also be calculated by a person skilled in the art by using measurements such as Live/Dead assay (Invitrogen UK), LDH assay (Promega UK), cell count method, immunocytochemistry staining, collagen assays (e.g Sircol Collagen Assay Kit, Biocolour UK), statistical analysis, and any other methods known to a person skilled in the art.

The hydrogel of the invention may be provided with one or more factors which influence the viability, growth, proliferation and/or differentiation of the encapsulated cells. Examples of such factors include growth factors, differentiation enhancing agent, extracellular matrix proteins, glycosaminoglycans and small molecule inhibitors. A growth factor is a protein, preferably but not necessarily a naturally occurring, which is capable of regulating one or more cellular processes, for example proliferation, migration, survival and differentiation. Examples of growth factors which may be used in the present invention include, but are not limited to, fibroblast growth factor (FGF), epidermal growth factor (EGF), neural growth factor (NGF), insulin-like growth factor (IGF), interleukin (IL), interferon (IF), platelet derived growth factor (PDGF), transforming growth factor (TGF), hepatocyte growth factor (HGF), tumour necrosis factor (TNF) and colony stimulating factor (CSF), vascular endothelial growth factor (VEGF) and bone morphogenic proteins (BMP). Using such factors, the hydrogel may be used for directing cell proliferation or differentiation, maintaining or altering cell behaviour, and affecting cell survival.

Suitable conditions for maintaining the gel for cell culture are preferably biologically acceptable conditions, for example as defined above in terms of ionic strength, pH and temperature.

Preferably, maintaining the hydrogel requires washing the hydrogel periodically, to ensure it remains stable and hydrated, the temperature remains stable, and any required supplements remain available to the encapsulated cells. Washing the hydrogel preferably comprises exchanging the media with fresh media one or more times during culture of the cells. Preferably, the media is the same media as that mixed with the first liquid in the method of preparing the gel, although this does not have to be the case and any suitable media, as defined herein, may be used. Preferably, the media is exchanged with fresh media an optimum number of times to maintain cell survival, allow cell growth, and provide hydrogel stability.

By washing is meant exchanging the media in which the hydrogel is present. Preferably, the media used to wash the hydrogels is of a predetermined, uniform volume. Preferably, the same type of media is used for each wash, although it is appreciated that different media may be used at different washes. Preferably, the media for washing is the same as the media used to prepare the hydrogel. It is appreciated that the washing media may be different to that used to prepare the gel. Preferably, the concentration of media is also uniform between washes.

The optimum number of media exchanges for a hydrogel may be determined by a method comprising the steps of:

i) preparing a plurality of gels encapsulating a cell, as defined herein;

ii) a gel setting, adding a volume of media onto each gel surface;

iii) for a first batch of gels, washing with media at first predetermined frequency; for a second batch of gels, washing with media at second predetermined frequency; and for any further batch of gels, washing with media at further predetermined frequency;

iv) extracting the cell from the gel;

v) counting the cells and determining the optimal number of washes needed to enhance cell culture within the gels.

Preferably, for the first batch of gels, the media is exchanged once, preferably after up to 0.5 days, preferably up to 2 hours, preferably up to 1 hour, preferably up to 30 minutes, more preferably up to 15 to 30 minutes after gel formation (setting). Preferably, for the second batch of gels, the media is exchanged a plurality of times, preferably between 2 and 4 times, preferably 3 times. Preferably the media is exchanged at intervals of up to 0.5 days, 2 hours, 1 hour, 30 minutes or 15 to 30 minutes. Preferably, for the third batch of gels, the media is exchanged a plurality of times, preferably between 3 and 7 times, preferably 5 times, at intervals of up to 0.5 days, 2 hours, 1 hour, 30 minutes or 15 to 30 minutes. More preferably, these media changes are performed for an initial period of time, for example between 0 and 3 days, followed by a period of cell culture with daily media exchanges.

Preferably, the volume of media used to wash the hydrogels depends upon the size of the hydrogel, but typically may be in the range of 0.1 to 10 ml, preferably 200 to 600 μl, preferably 200-400 μl.

In the context of the present invention where referring to the age of a hydrogel, this is relative to the time at which the hydrogel is made (i.e. the second solution sets to form a gel). For the purposes of the present invention, intervals of 24 hours from the setting of the gel are referred to as days. Thus, day 1 is 0 to 24 hours after setting of the gel; day 2 is 24 to 48 hours after setting of the gel; day 3 is 36 to 48 hours after setting of the gel, and so on. In the same fashion, 0.5 days is 12 hours from setting of the gel.

It is envisaged that the cells may be extracted from a hydrogel after a defined period of cell culture, either for future use, or to be introduced into a second or further hydrogel for further cell culture. Thus, the present invention provides a method of extracting cells from a hydrogel comprising:

1) physically disrupting the gel;

2) separating the cells from the media by centrifugation or sedimentation;

3) removing the media from the cells.

Physical disruption of the hydrogel includes, for example, shaking, stirring, pipetting and any other suitable means which disrupts the assembly of the peptides into nanofibres sufficiently to destroy the three-dimensional peptide scaffold, without causing undue cell damage, for example heating. In a preferred embodiment of the invention, mechanical disruption is used to liquefy the hydrogel. Other non-physical methods may be used, preferably those which do not adversely affect the viability of cells supported by the hydrogel.

The invention provides a method whereby extracted cells are mixed with media, preferably the same media as that used in the preparation of a hydrogel, and the media comprising the cells is then mixed with a first solution to provide a second solution for the preparation of a further hydrogel.

Where the cells are passaged into a second or further hydrogel a correction must be made regarding the dilution of the cells, to ensure the correct final cell concentration. In an embodiment, the method comprises i) providing two or more hydrogels, preferably prepared by step 1) to 3) of first aspect; ii) liquefy a first gel, and encapsulate cells, preferably as defined in step 5) of the first aspect; iii) maintain under conditions suitable for the formation of a hydrogel; iv) liquefy the gel and extract cells therefrom; iv) liquefy a second gel; v) add cells thereto; and vi) repeat steps i) to v) any number of times, depending on number of passages required.

The present invention has the advantage of allowing cell extraction, and gel to gel passage without risk of loss of cells when placed in a new gel. This is a feature of using pre-formed hydrogel, and also the optimisation method which allows for reliability and increased reproducibility of the gel. The cells grown in the hydrogel may be extracted with maximal viability (see notes) eg for passage into further gels or for further culture; with minimal carry-over of gel for cell counting, for example by FACS, or cell-derived material may be extracted for PCR or other analysis. The gels of the invention enable the cells (or cell-derived material) to be extracted in a variety of purities—i.e., the amount of gel which is present, or the degree to which the cells can be extracted without gel present.

Extraction may be performed by any suitable means, including for example mechanical disruption or enzymatic degredation. Where cells are required for further culture or use as live cells, it is desired to use a cell friendly extraction method such as physical or mechanical disruption. Preferably, at least 25, 50, 75, 80, 90 or 95% cells are extracted from a hydrogel. Depending upon the method used, the viability of the extracted cells may be 10% of cells, 20, 30, 40, 50, 60, 70, 80, 90 or 95% of extracted cells. The method used will depend upon the final purpose and use of the cells, and the degree to which cell viability is necessary.

Extracted cells may be used for analysis, for example of cellular material including for example nucleic acid, proteins and sugars. For example, analysis may include flow cytometry, to produce a single cell suspension or for further culturing for example in another gel; or for use as a therapeutic. For example, an extracted cell or culture of cells may be introduced into a subject, for example by any route such as intravenous, subcutaneous, oral, percutaneous, intramuscular, or surgical methods. An extracted cell may be used to repair or supplement an organ, tissue or body structure. An extracted cell may be used to provide or supply a therapeutic agent. The cells may be genetically modified cells.

Organs or bodies include brain, nervous tissue, bladder, oesophagus, fallopian tube, heart, intestines, gall bladder, pancreas, liver, kidney, lung, ovaries, prostrate, spinal cord, spleen, stomach, testes, thymus, thyroid, trachea, ureter, urethra and uterus.

A liquefied hydrogel comprising cells may be used in a method of tissue repair or regeneration. Preferably, such a method may be carried out, although it is envisaged that the hydrogel may be used in the repair or regeneration or indeed making of tissues, in vitro. In such a method, the liquefied hydrogel comprising cells is introduced into the tissue by any suitable means, for example injection although it is envisaged that other methods of introduction may be used, as selected by the medical practitioner.

Thus, the hydrogel is capable of being produced and encapsulating cells, liquefied, and introduced into tissue where it re-forms into a hydrogel in situ under suitable biological conditions, preferably as described herein. This method allows the hydrogel to fill the tissue regeneration site and provides a support structure which the cells can populate and integrate with surrounding tissue.

This aspect enables the hydrogel of the present invention to be used in the treatment of a variety of medical conditions such as tissue degenerative disorders such as neurodegenerative, intevertabral disc disorders, cartilage or bone degenerative disorders including for example, osteoarthritis, osteoporosis, liver degenerative disorders, kidney degenerative disorders, muscle atrophy. In addition, the invention is suitable for the treatment of medical conditions which are the result of tissue damage or tissue loss. Such conditions include, for example, wounds and injuries, as well as the above mentioned tissue degenerative disorders. Wounds may be chronic, abrasive, pressure induced such as bed sores or lesions, penetrative, such as a cut or stab wound, or internal. Examples of wounds include abrasive wounds, such as burns, pressure wounds, such as sores and ulcers, penetrative wounds such as cuts or stab wounds. Tissues which may be in need of repair using a hydrogel of the present invention include epithelial, connective, muscular or nervous tissue. Preferably, the tissue is cartilage, for example articular, fibrous, semilunar, hyaline, costal, thyroid or elastic cartilage. Organs which may be sites of tissue damage include bladder, brain, fallopian tubes, heart, intestines, gallbladder, kidney, liver, lungs, oesophagus, ovaries, pancreas, prostate, spinal cord, spleen, stomach, testes, thymus, thyroid, trachea, ureter, urethra and uterus. The present invention may also be used for correcting injury or defects in joints, such as knees, his, elbows, neck, wrists, ankles, toes, fingers and elbows and shoulders.

A hydrogel of the invention may preferably comprise one or more of the following independently selected properties:

i) is capable of a liquid-gel-liquid transition or a gel-liquid-gel transition

ii) is capable of maintaining cells in culture for at least 1 hour, 2, 3, 4, 5, l 6, 7, 8, 9, 10, 12, 15, 20 or 24 hours or at least 1, 2, 3, 4, 5, 6, 7, 8, 9, 10, 11, 12, 13, or 14 days, preferably in 3D;

iii) is capable of supporting proliferation of cells, preferably in 3D;

iv) is capable of enabling extraction of cells;

v) is capable of supporting transition of cells to a second or further hydrogel;

vi) is capable of maintaining a three dimensional structure;

vii) comprises nanofibers of between 20 nm and 5 nm in width; and

viii) exhibits hydrogel stability for at least 1, 2, 3, 4, 5, 6, 7, 8, 9, 10, 11, 12, 13, 14, 21, 25, 28 or 31 days;

ix) is produced by a method of the invention.

The physical properties of a hydrogel may be altered depending upon its purpose by changing the length of the peptide (increasing the length may increase rigidity); altering the hydrophobicity (increasing the hydrophobicity increases rigidity), changing the concentration of the peptide; altering the media type used, etc. These factors may affect the brittleness, fragility, ductility, elasticity, stability and rigidity of the gel.

A kit for making a hydrogel of the invention, comprising a peptide comprising an amphiphilic portion as described herein, and instructions for carrying out the method of the invention. A kit may comprise one or more of amphiphilic peptide, as described herein, a predetermined amount of base, a predetermined amount of cell culture media, instructions for gel formation and encapsulating cells therein; instructions for cell extraction; instructions for optimising peptide; for liquefying gel; encapsulating cells and reforming gel; a cell population, a delivery device, a vessel for mixing the peptide composition and base, or liquefied gel and cells.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Examples

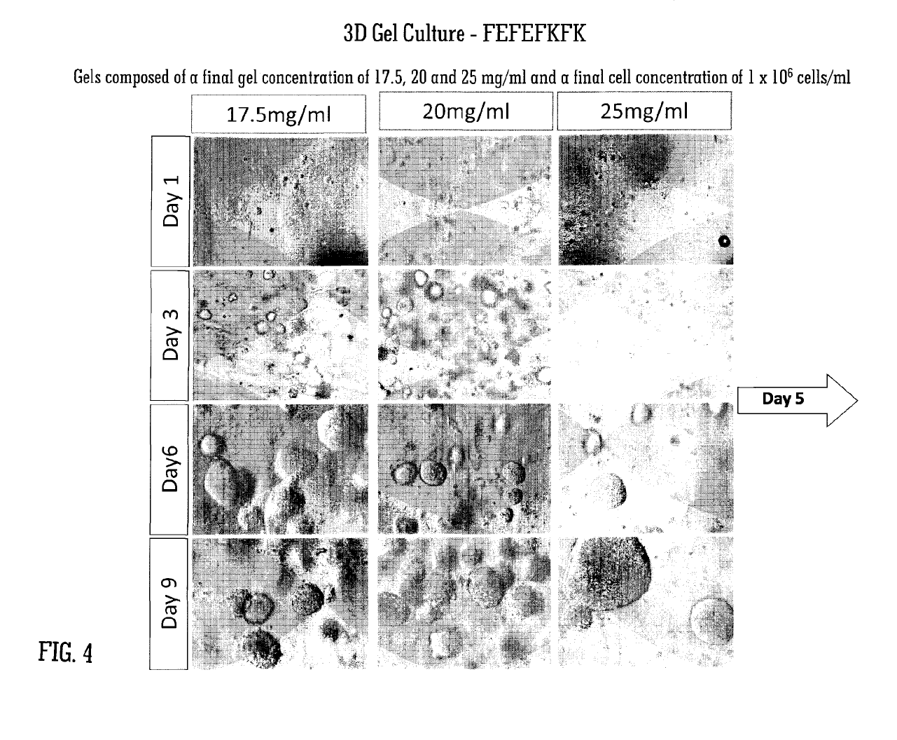

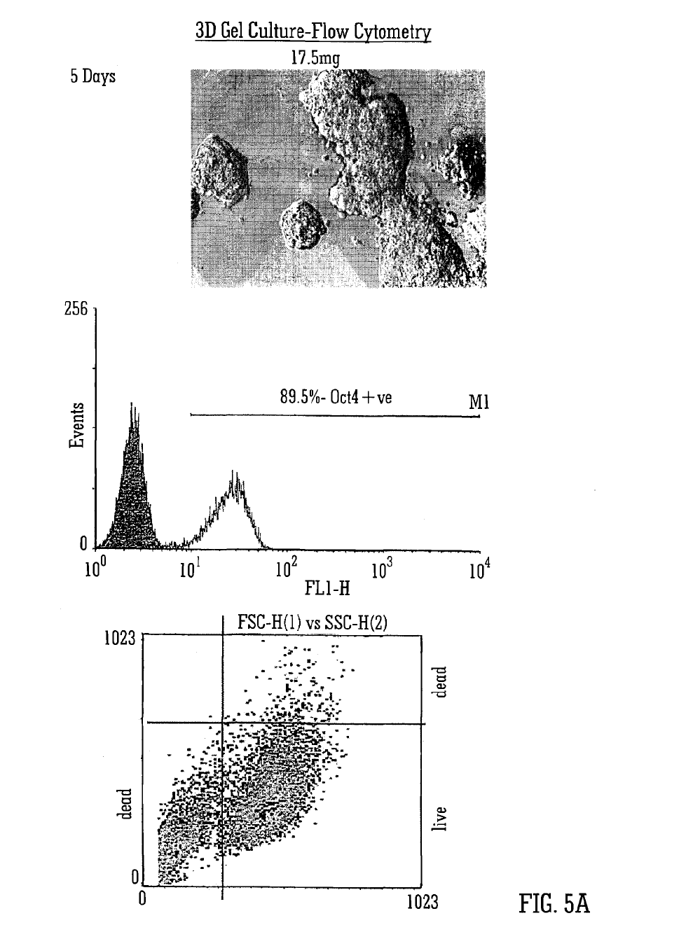

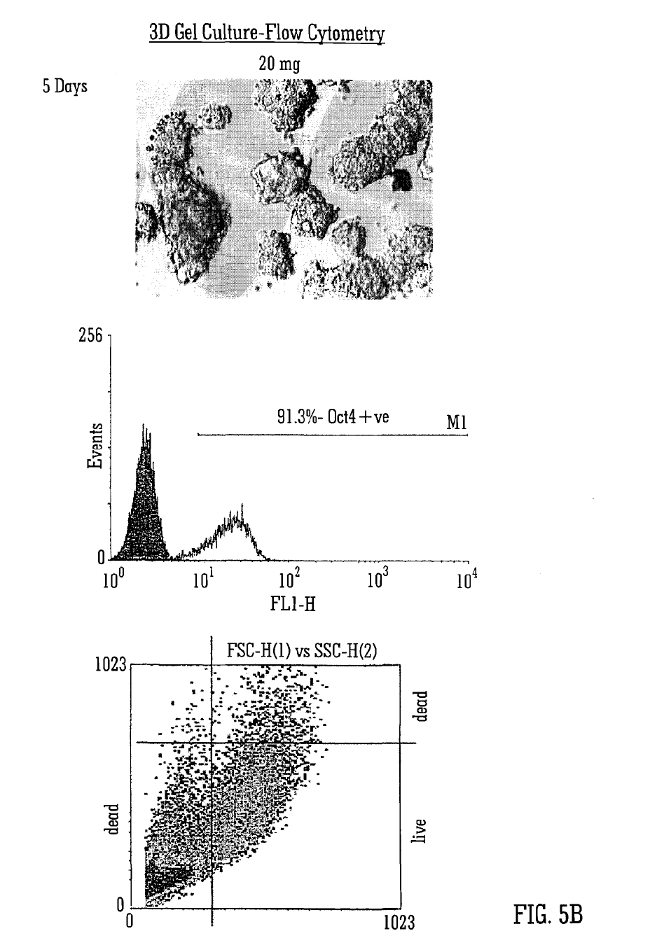

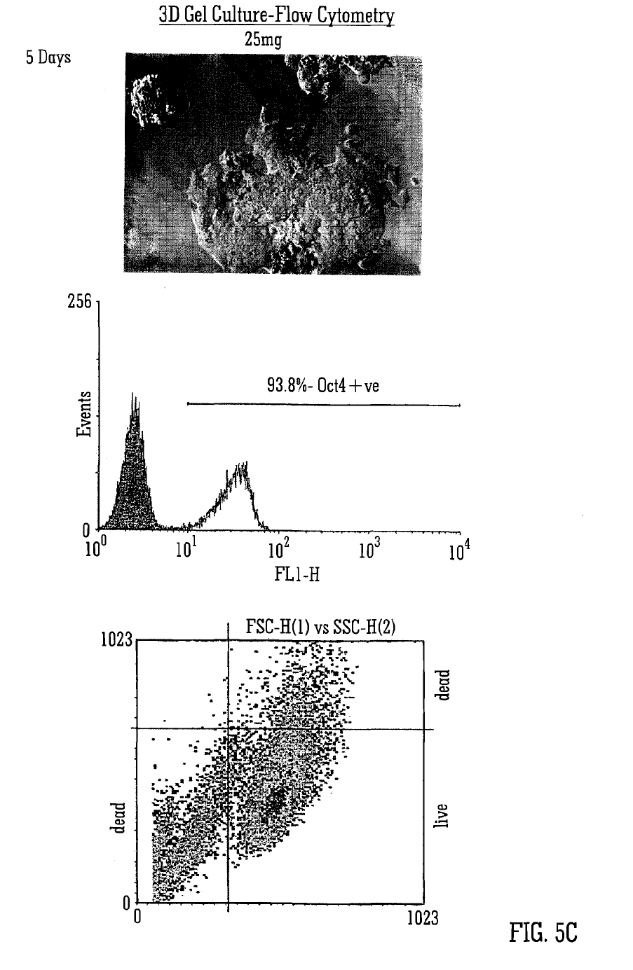

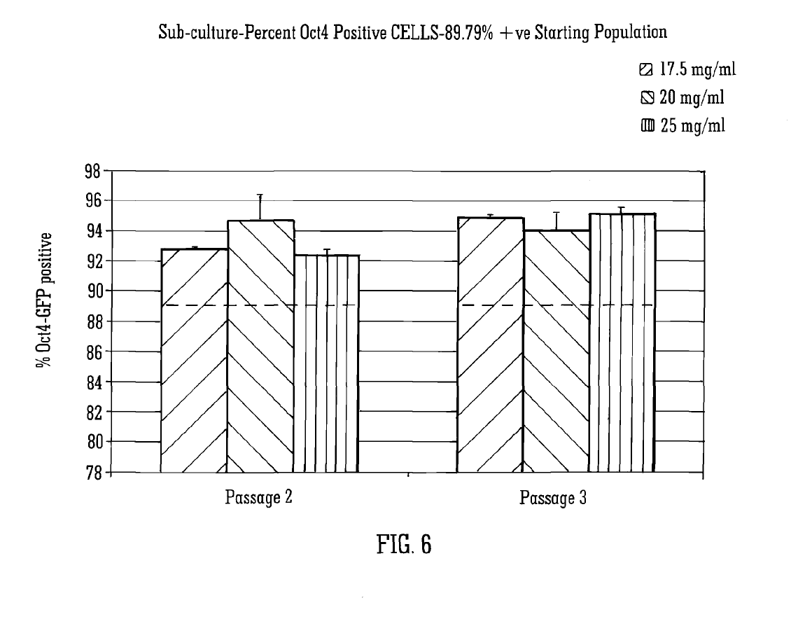

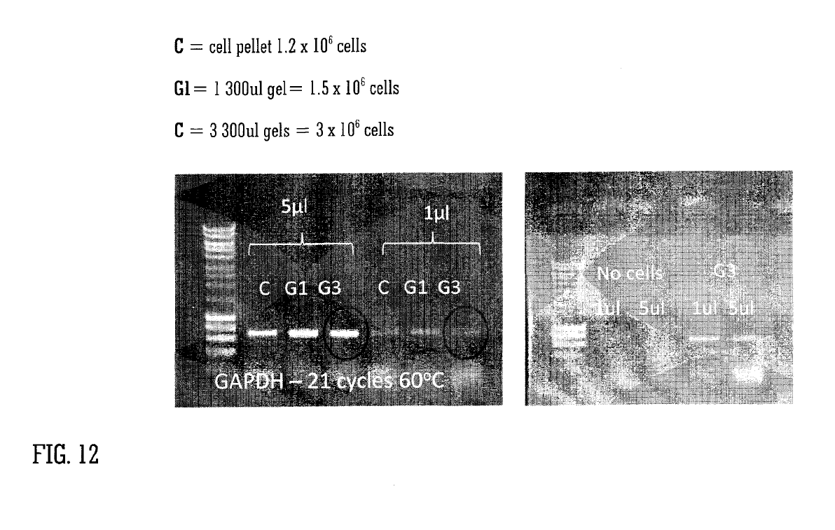

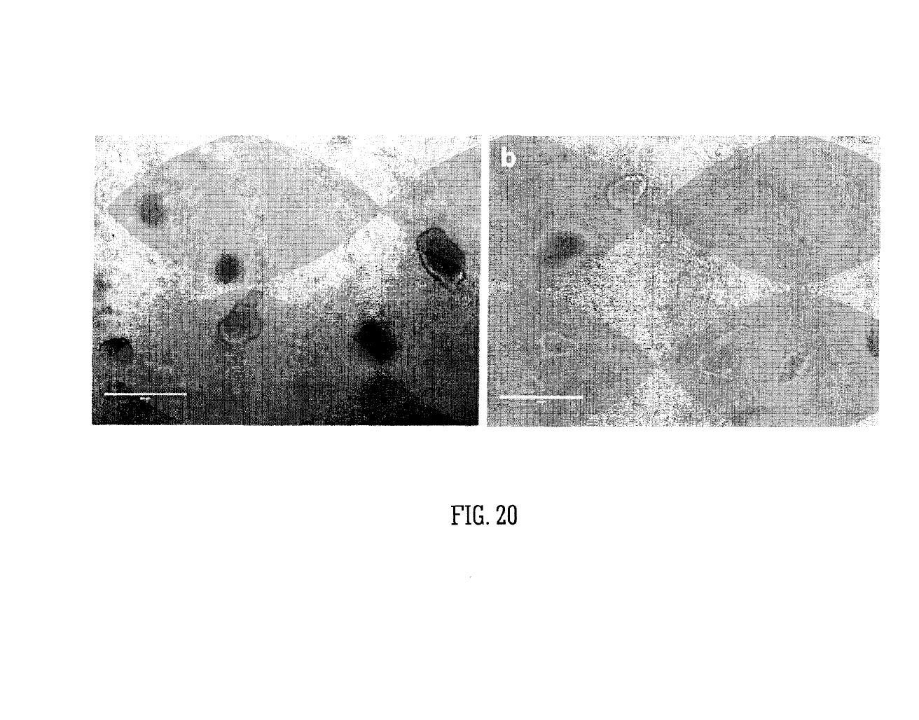

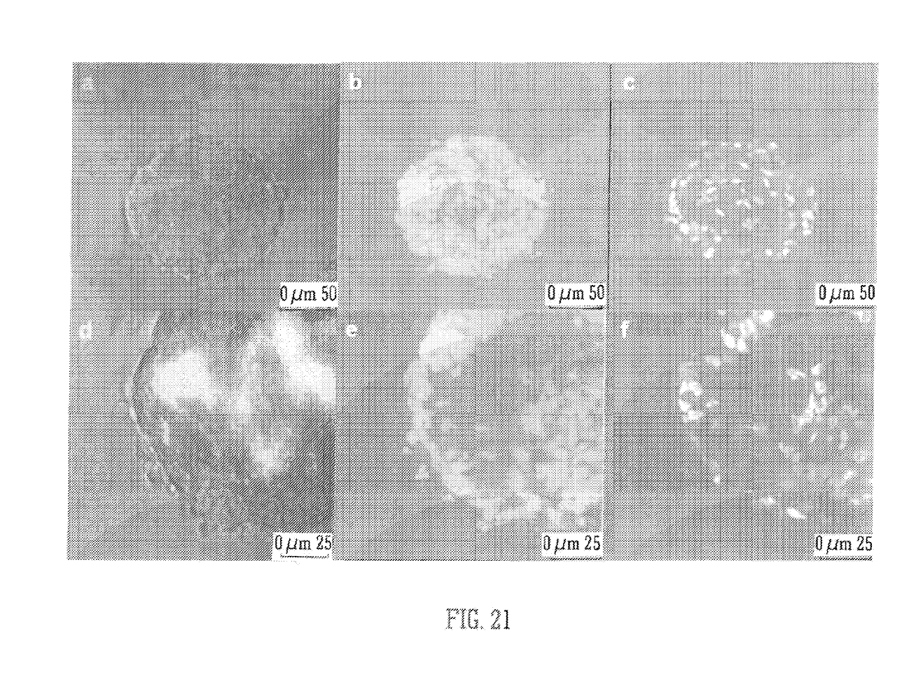

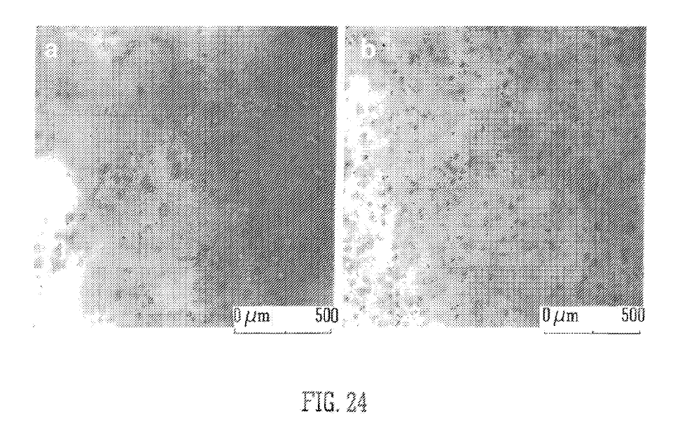

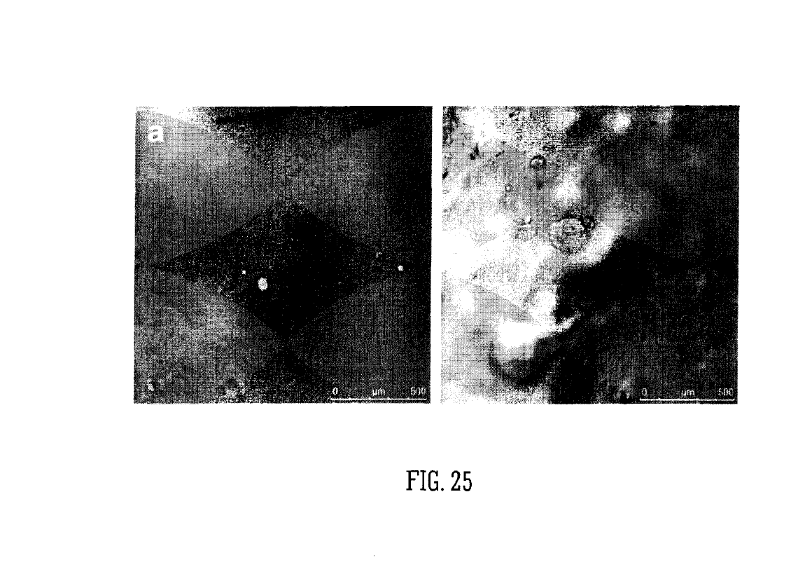

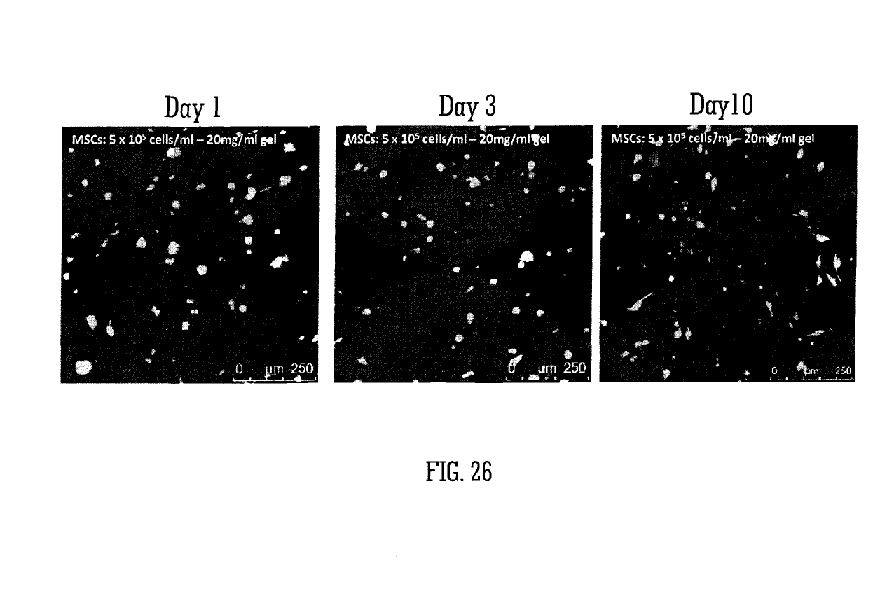

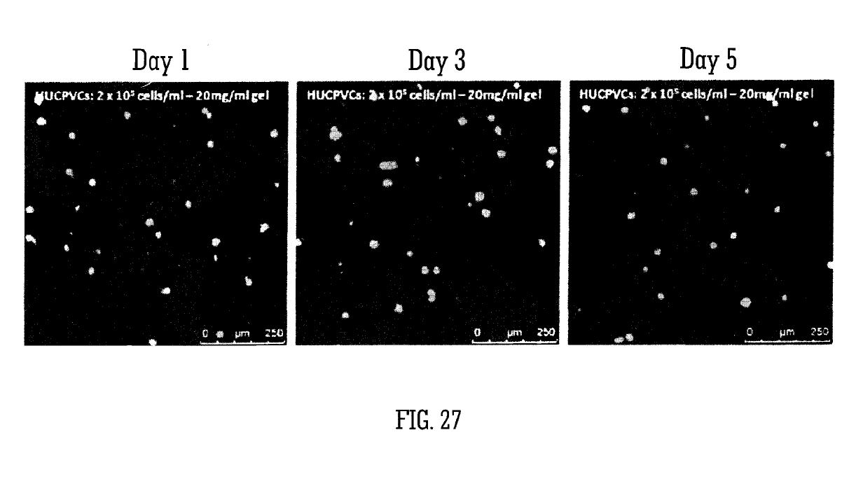

Using mouse embryonic stem (mES) cells, human embryonic stem cells (hES) and human (adult) mesenchymal stem cells (MSCs both bone marrow derived (called MSCs hereafter) and umbilical cord perivascular cells (HUCPVCs)) the present inventors have been able to demonstrate that the cells can be encapsulated in the gels and maintained in culture for at least 5 days. For mES culture after 5 days the cells grew as large clusters (increased cell numbers indicating good cell proliferation, FIGS. 4-11). hES cells were seeded either as single cells or dissociated colony pieces supporting the proliferation of viable cells with some evidence of differentiation (FIGS. 20-25). Human ES cells could be cultured for at least 5 days in the gels. Adult human stem cells (MSCs) remained viable within the gels for at least 10 days (FIG. 26). A method was then optimised for release of cells from the gels and sub-culture of cells into fresh gels, allowing continuous passage of the cells within the gels (FIG. 6 demonstrates this for mES). Human adult stem cells (HUCPVCs) could also be sub-cultured with retention of cell viability (FIG. 27). With mES cells, the inventors were able to demonstrate that the proportion of Oct4 positive cells within the population (an indicator of the ‘stemness’ of the cells) increased as the cells went through the sub-culture regime indicating that the gel culture conditions were favourable to retention of stem cell characteristics. The morphology of the human ES cells (typically after 5 days of culture in the gels) suggests that they are not fully retaining their self-renewal characteristics and some cells are undergoing differentiation (FIGS. 21 and 23). The human adult stem cells (MSCs) initially seeded in the gels as single rounded cells, shift to a more spread morphology by day 10 of culture (FIG. 26). As well as sub-culture, the inventors have optimised a method the for extraction of cells with minimal carry-over of gel debris from the gels for analysis by flow cytometry (FIG. 5). In addition, a method for the efficient extraction of mRNA from the cells grown within the gels was optimised (to enable assay of cell behaviour by PCR, demonstrated in FIG. 12).

A) Gel Optimisation

Adjusting pH

The amount of NaOH needed to achieve the correct pH varies with each peptide batch. Therefore, it is necessary to pre-determine the amount of NaOH needed before adding cells to the gels.

Prepare one or two gels. Weigh out the appropriate amount of FEFEFKFK peptide into 15 ml tubes. The final volume of the gel will be approximately 1.25 ml therefore, if a 20 mg/ml gel is required weigh 25 mg. If 25 mg/ml gels are needed then weigh 31.25 mg.

Centrifuge the tubes for 1 minute at 4000 rpm to pellet the peptide to the bottom of the tube.

Add 800 μl sterile dH₂O and vortex. Recollect the peptide to the bottom of the tube by spinning for 1 minute at 700 rpm.

Melt/dissolve the peptide for 2 hours at 95° C.

Add 100 μl 0.5M NaOH to all gels. When adding the NaOH gently stir into the centre of the gel.

Add 10 μl 0.5M NaOH sequentially, mixing the gel thoroughly with a vortex after each addition until a largely clear gel has formed (some cloudy aggregates may be present).

Add 100 μl 10× Dulbecco's Phosphate Buffered Saline (DPBS, without magnesium and calcium, PAA). Stir into the centre of gel when adding and vortex thoroughly. If the gel is not clear continue to add 10 μl 0.5M NaOH until the gel clears.

Spin the gels at 4000 rpm for 1 minute to remove bubbles.

Transfer the gels back to the water bath/hot-block/oven to incubate for 16-18 hours (overnight) at 95° C. This improves mixing of the gel with the NaOH and DPBS, increasing gel reproducibility.

Vortex the gel and let it cool down.

Check the pH of the gels using a pH probe. The optimum pH of the gel should be between 9.0-9.4.

If the gels are cloudy and the pH is <9, add 0.5M NaOH 5 ul at a time—mixing well between each addition until the pH is within these limits.

If the pH is >9.4, persevere with the gel until step 21. If the gel is unstable and outside the pH range described in step 21 after media addition, prepare new gels taking care to add less 0.5M NaOH as described in step 22.

Centrifuge at 4000 rpm for 1 minute. UV Sterilise the gels for 30 minutes in a class II tissue culture hood.

Add 250 ul media to the gels (minus cells). Pipette the media on top of the gel and gently mix by stirring the media in with the pipette tip. Create a more homogenous mixture by pipetting gently up and down, taking care not to create bubbles.

Excess bubbles can be removed by spinning for 2 minutes at 1500 rpm.

Transfer 300 μl of the gel into a 12 well transwell insert plate or a standard 24 well plate.

Leave to set for ten minutes at 37° C. and then gently add media.

Incubate the gel in an incubator at 37° C., 5% CO₂ gently exchanging the media every day.

Assess the pH of the remaining, unplated gel after media addition using a pH probe. For best setting the final pH after media addition should be between pH8.8-9.5. A stable gel should remain intact for over five days.

If the gel was cloudy and unstable, follow the same protocol as described above but add 5-20 μl more 0.5M NaOH than previously. Repeat until the gel with media is clear, stable and within the desired pH range. If the gel was clear but unstable during culture, follow the same protocol as described above but add 5-20 μl less 0.5M NaOH until the gel is stable and at the lower limits of the desired pH range.

Gel Washes

Once the optimum amount of NaOH has been determined it is necessary to establish whether and to what extent the gel needs to be washed after setting to encourage optimal cell survival.

Prepare three gels (labelled 1-3) as described in section B, 1-17, plating all the gels in the wells of a 12 well transwell insert plate or a standard 24 well plate. There should be approximately four complete 300 μl gels per 1.25 ml gel (labelled A-C).

Once the gels have been allowed to set for 10 minutes at 37° C., add media.
Exchange the media after 15-20 minutes on gels 1A, 2A, and 3A
   Exchange the media three times every 15-20 minutes on gels 1B, 2B and 3B.
   Exchange the media five times every 15-20 minutes on gels 1C, 2C, 3C.

After the initial media change, culture the cells within the gel for five days changing the media every day.

At day five of culture, extract the cells as described in section D, creating a single cell suspension will allow you to count the cells within the gels and determine the optimal number of washes needed to enhance cell survival within the gels.

B) Gel Preparation Protocol

Weigh out the appropriate amount of FEFEFKFK peptide into 15 ml tubes. The final volume of the gel will be approximately 1.25 ml therefore, if a 20 mg/ml gel is required weigh 25 mg. If 25 mg/ml gels are needed then weigh 31.25 mg.

Centrifuge the tubes for 1 minute at 4000 rpm to pellet the peptide to the bottom of the tube.

Add 800 μl sterile dH₂O and vortex. Recollect the peptide to the bottom of the tube by spinning for 1 minute at 700 rpm.

Melt/dissolve the peptide for at least 2 hours at 95° C.

Add the amount of 0.5M NaOH predetermined in section A followed by 100 μl 10×DPBS. Alternatively the equivalent amount of 1.5M NaOH can be added. However, this must be pre-mixed with dH2O to get a final volume of 100 μl before adding to the gel in order to aid mixing.

Spin the gel for 1 minute at 4000 rpm and transfer the gels back to the water bath/hot-block/oven to incubate for at least 4 hours at 95° C.

After this incubation period, the gels can be stored for up to one month at 4° C.

Once the gels are ready for cell seeding UV sterilise the peptide in a class II tissue culture hood for 30 minutes at room temperature.

Add cells to the gels in a 250 μl volume of media. Pipette the cell suspension on top of the gel and gently mix by stirring the cells in with the pipette tip.

Create a more homogenous mixture by pipetting gently up and down, taking care not to create bubbles.

Excess bubbles can be removed from the gel/cell mixture by spinning for 2 minutes at 1500 rpm.

Plate 300 μl of the gel into a 24 well plate or more preferably a 12 well plate cell culture transwell insert. Allow the gel to set for 10 minutes at 37° C. in a cell culture incubator.

Gently add media and exchange the media as determined in section A) Gel Optimisation.

The above method was used to encapsulate mouse ES cells (FIGS. 4-11) and MSCs (FIG. 26). The above protocol was used to prepare gels for use with hES cells (FIGS. 20-25).

C) Cell Extraction Protocol for Subculture

Add 1 ml of media to the top of the gel. Pipette the media on the surface, using enough force to disrupt the gel within the well. Pipette up and down, breaking up the gel as much as possible.

Transfer the gel/media suspension to a 15 ml tube. Wash out the well with another 1 ml media and transfer this to the same 15 ml tube.

Centrifuge at 1000 rpm for 5 minutes.

After centrifugation there should be a deposit of gel debris at the bottom of the tube. Aggregates may be visible within the suspension—they reside within the bottom 100 μl.

Carefully remove clear media at the top of the gel debris, leaving the cloudy gel deposit intact.

Using the above method, Oct4-GFP mouse ES cells and human adult stem cells were successfully subcultured within the gel (FIG. 6 and FIG. 27).

D) Flow Cytometry

Method of Extraction for Immediate Flow Analysis

Remove the gel from the insert and place into a 15 mL falcon tube.

Add 1 mL of growth media and mix thoroughly by vortexing

Add 4 mLs of media and vortex

Centrifuge the samples at 1000 rpm for 5 minutes

Remove the media leaving the gel, check the bottom of the tube—a cell pellet should be visible underneath the gel layer. If it is not repeat steps 2 to 4.

Carefully remove the gel layer leaving the cell pellet behind.

Add 5 mL of PBS and mix by pipetting

Centrifuge the sample at 1000 rpm for 5 minutes

Remove the PBS and any remaining gel fragments

Add 1 mL of TrypLE™ and place in the water bath for 5 minutes—shaking the tube a few times.

Mix the samples by pipetting

Leave the sample to settle for 1 minute allowing any remaining gel fragments to fall to the bottom of the tube. Most of the cells should still be in suspension at this point.

Carefully remove the cell suspension leaving behind any gel fragments.

Place into a fresh 15 mL tube

Add 5 mL of centrifuge the sample at 1000 rpm for 5 minutes

Re-suspend in 500 μL and perform a cell count.

Check the cell suspension in the haemocytometer for any remaining gel fragments.

Re-Plating for Flow Cytometry

Dilute the gel deposit with 10 ml media and centrifuge again at 1000 rpm for 5 minutes.

Carefully remove clear media leaving the cloudy gel deposit intact.

Re-suspend the gel deposit in 3 ml media and plate into the well of a twelve well plate. If it is mouse embryonic stem cells that are being extracted, ensure the twelve well plate is pre-gelatinised with 2 ml 0.1% gelatin in dH₂O overnight at 4° C., ensuring that excess gelatin is removed before plating.

Incubate the cells for 16-24 hours in an incubator set at 37° C. at 5% CO₂.

Once the cells are attached, remove the excess gel debris containing aggregates that have not attached to the plate.

Wash the attached cells with 2×1 ml DPBS and incubate with 100 μl trypsin (Invitrogen) or dissociation buffer (sigma) at 37° C., 5% CO₂ for 3 (trypsin) or 5-10 (disassociation buffer) minutes.

Detach cells by washing off with 300 μl media and transfer the resulting cell suspension, which should have a total volume of 400 μl, to a flow cytometry tube containing 100 μl 5% formaldehyde. Mix the suspension and transfer the tube to 4° C. for storage.

The above method was utilised to analyse Oct4-GFP mouse ES cells extracted from FEFEFKFK peptide gels (FIG. 5).

E) Creating a Single Cell Suspension

Disrupt the gels as described in section C

Dilute the gel deposit in 10 ml cell media.

Centrifuge at 1000 rpm for 5 minutes.

If a clear cell pellet is visible, remove the clear, excess cell media and excess gel deposit, leaving behind the intact cell pellet.

If a cell pellet is not visible, remove the clear media fraction only, resuspend the gel deposit in 1 ml of media, vortex and re-centrifuge.

Re-suspend the cell pellet in 1 ml trypsin or TrypLE™ and incubate in a water bath for 3-5 minutes at 37° C.

Pipette the solution up and down to break up any remaining cell aggregates.

Neutralise the trypsin or TrypLE™ with 1.5 ml media.

Centrifuge at 1000 rpm for 5 minutes.

Remove the clear media above cell pellet.

Resuspend the cell pellet in the desired volume of media.

Remove 10 μl and count the number of cells using a haemocytometer.

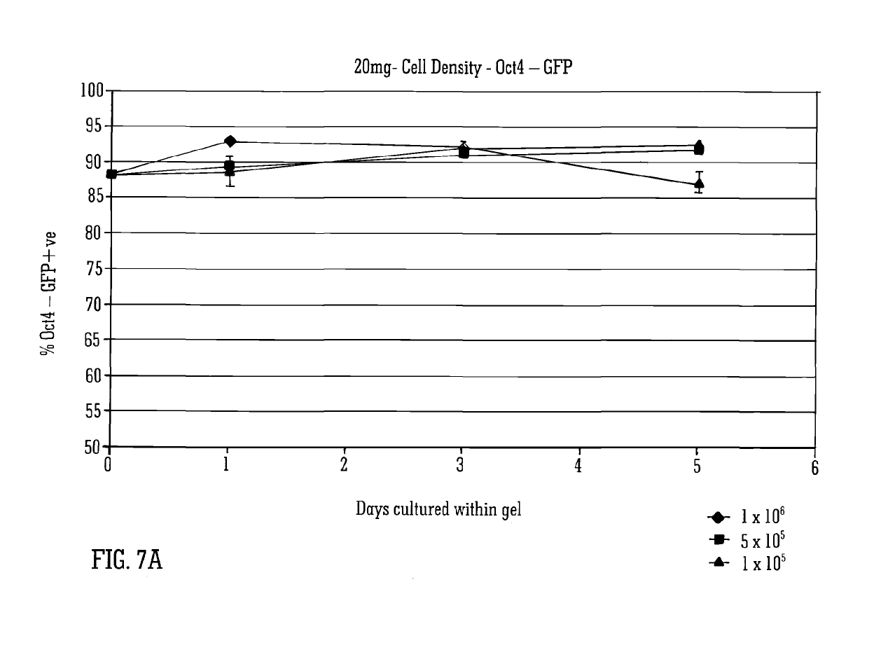

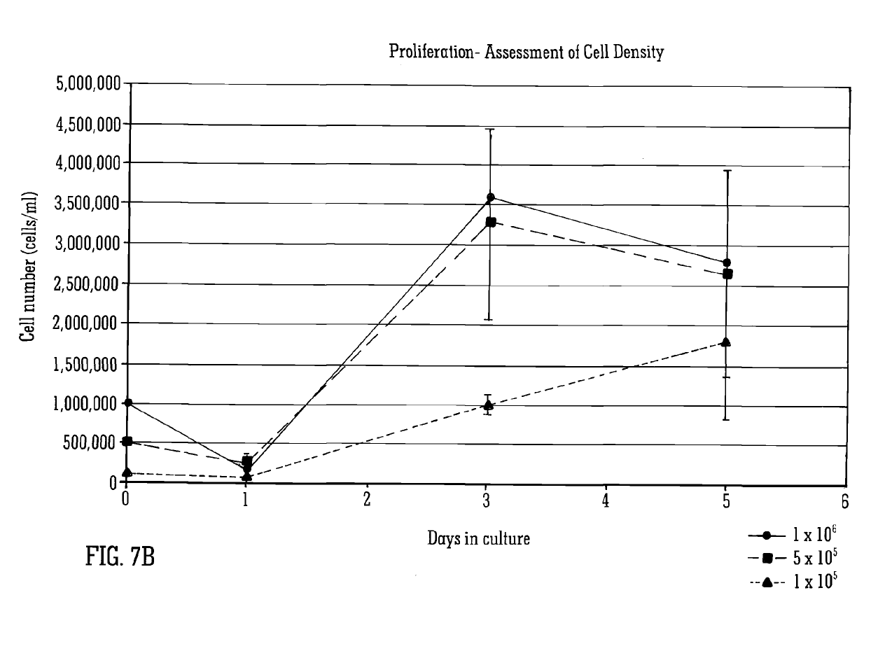

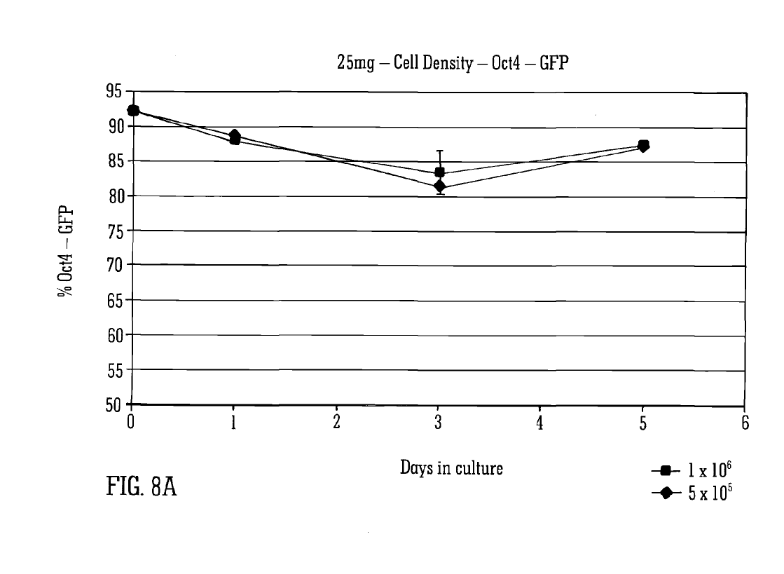

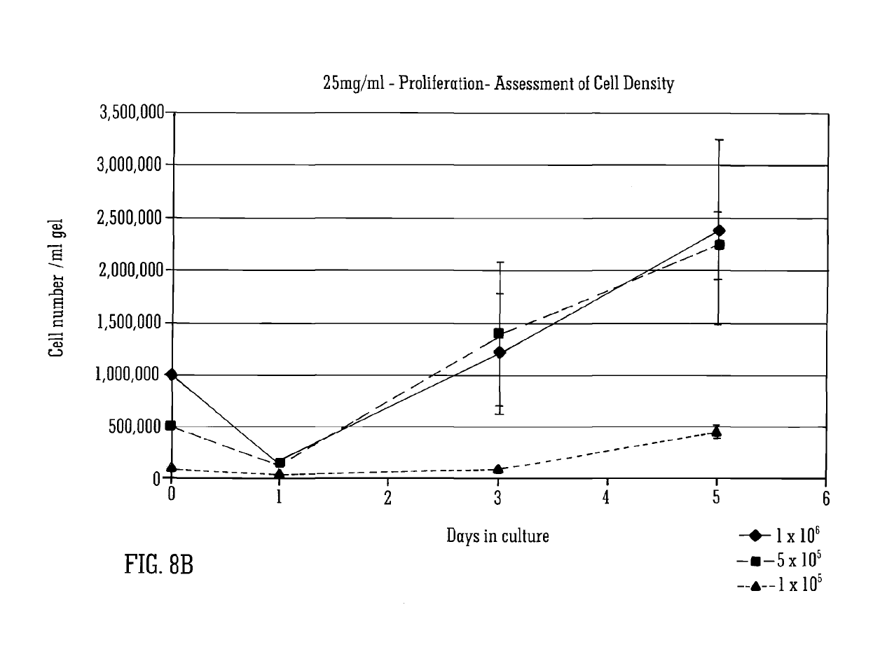

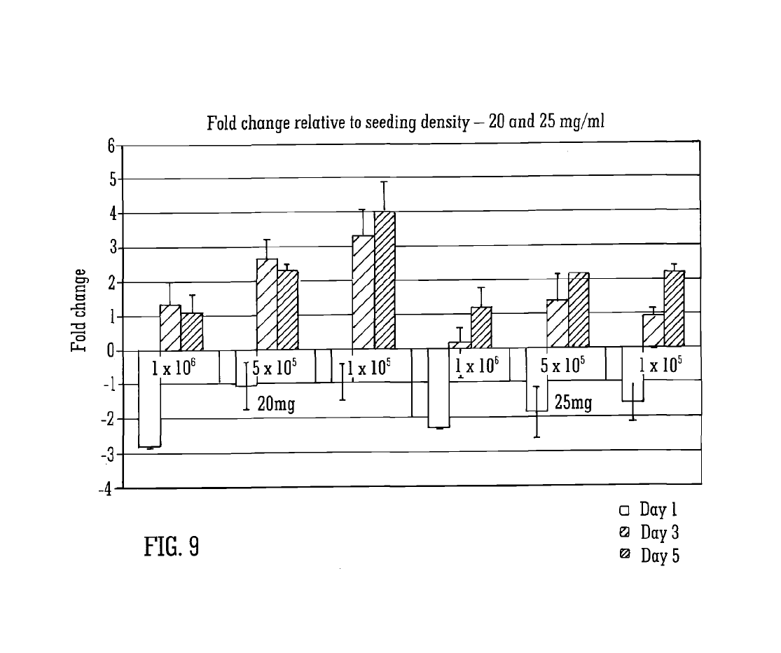

The above method was used to count cells extracted from gels allowing assessment of proliferation within the gels. This data is represented in FIG. 7, FIG. 8 and FIG. 9.

F) RNA Analysis

Re-suspend the gel in 1 ml media.

Centrifuge the suspension at 1000 rpm for 5 minutes. Remove the excess media leaving behind the intact gel deposit. Extract the RNA by treating the deposit as a cell pellet and following the protocol of your chosen manufacturer.

Using the above method, extracted RNA can be used in subsequent PCR analysis as depicted in FIG. 12.

G) Passaging into Fresh FEFEFKFK Gels

Prepare fresh gels by following section B.

Remove cells from old gels by following section E.

Create the correct cell concentration for seeding either by diluting the suspension further or re-centrifuging and re-suspending the gel deposit in a smaller media volume.

Mix the cell suspension (250 μl volume) into the freshly prepared first solution.

Using the above method, Oct4-GFP mouse ES cells and human adult stem cells were successfully subcultured within the gel (FIG. 6 and FIG. 27).

H) Transmission electron microscopy (TEM): The morphology and the width of the fibres within a self-assembled hydrogel may be investigated using a Tecnai 10 TEM operating at 100 eV (calibrated magnification of 43,600×) onto a Kodak SO-163 film. Images are subsequently scanned using a UMAX2000 transmission scanner providing specimen level increment of 0.366 nm pixel⁻¹. Carbon coated copper grids (400 mesh, Agar scientific) are glow discharged for 5 seconds and placed shiny side down onto a 10 μl droplet of diluted hydrogel for 10 seconds. Loaded grids are immediately placed on a 10 μl droplet of double deionised water for 10 seconds and then blotted. Washed grids are then placed on a 10 μl droplet of freshly prepared and filtered uranyl acetate solution (4% w/v) for 60 seconds for negative staining and then blotted continuously against double folded Whatman 50 filter paper.

I) Cryo-scanning electron microscopy (Cryo-SEM): Cryo-SEM is performed using a Philips XL30 ESEM-FG equipped with an Oxford Instrument Alto CT2500 cryo-transfer system. The sample prepared is placed between the rivets located on one end of the transfer rod and then snap frozen in liquid nitrogen before being transferred to the cryostat chamber. The temperature in the chamber is maintained at −150° C. The coated sample is then transferred to the microscope chamber. Images are captured with the microscope operating under high vacuum and with an accelerating voltage of 5 kV.

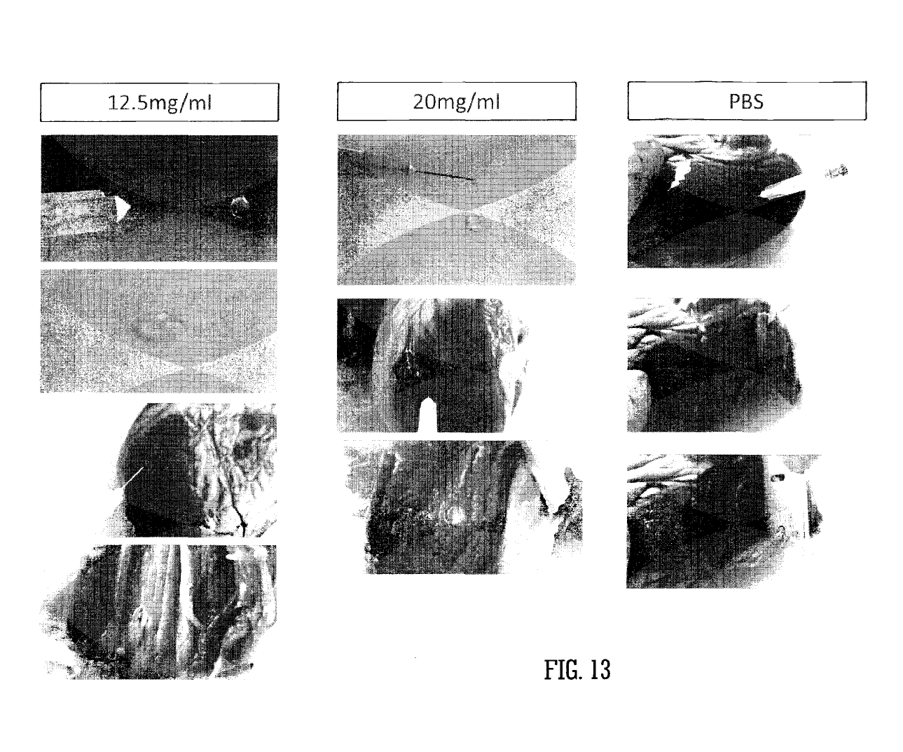

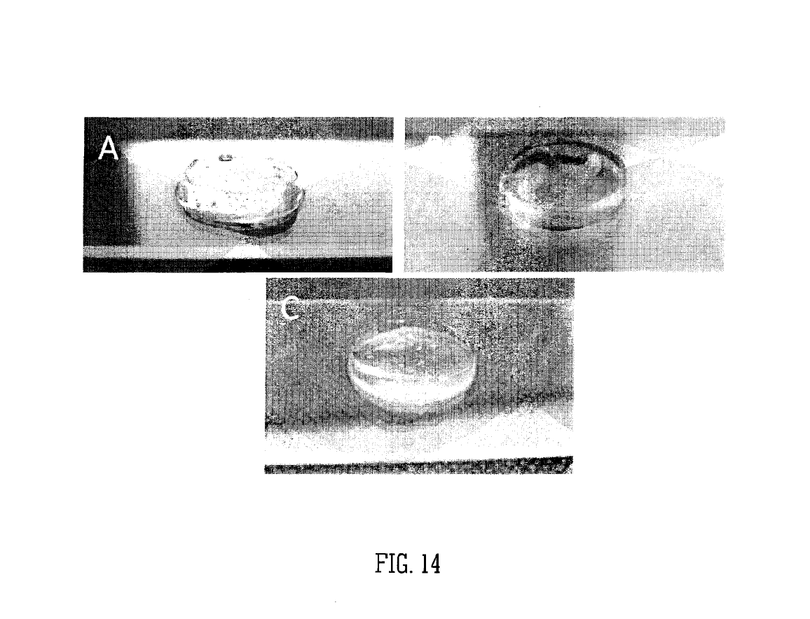

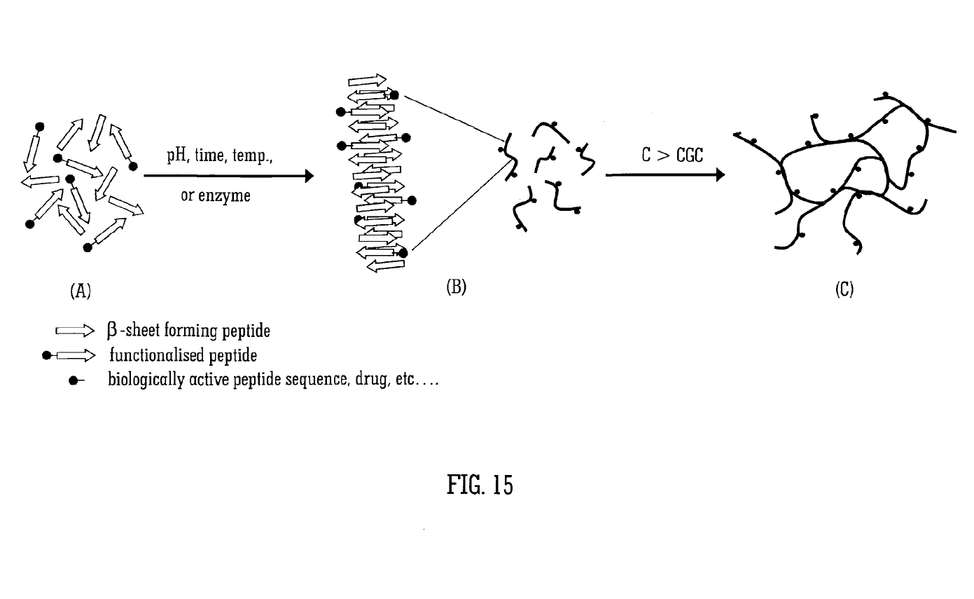

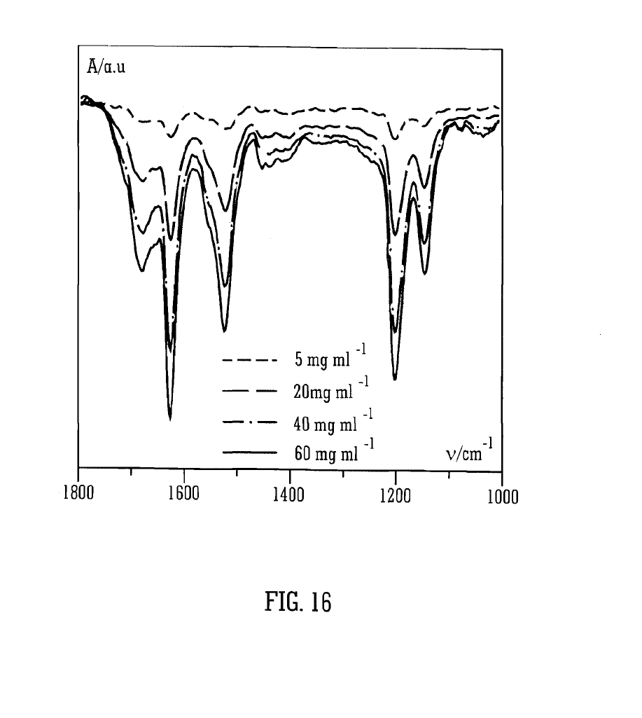

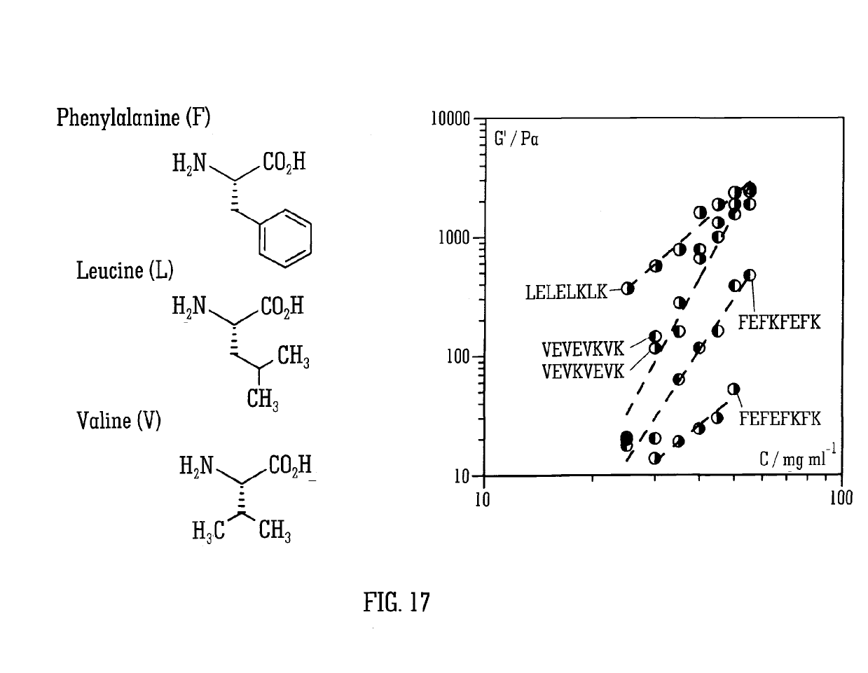

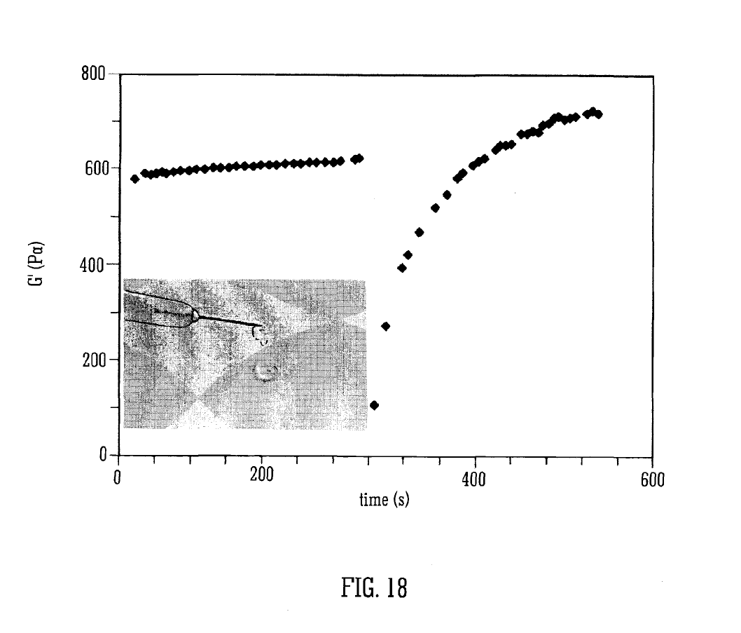

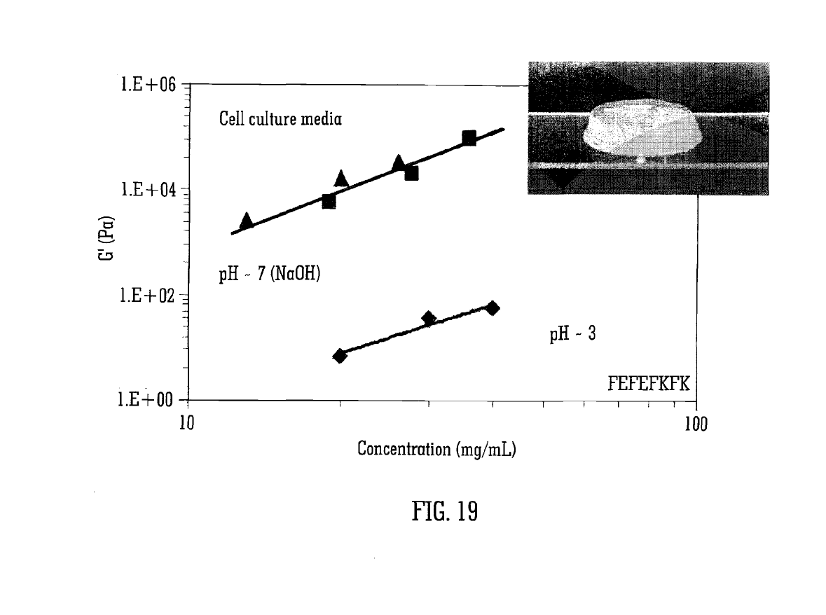

J) Rheology: The viscoelastic properties of a hydrogel are investigated on a Bohlin C-VOR 200 digital rheometer with 20 mm/2° parallel plate geometry. The elastic (G′) and viscous (G″) moduli of the hydrogels are recorded as a function of frequency sweeps between 0.1 and 100 Hz at 0.1% strain. A solvent trap is used to keep the hydrogels hydrated and the temperature was maintained at 25° C. Oscillatory tests are also carried out using cell seeded gels in 2D and 3D. For this experiment, all variables are kept constant and the temperature maintained at 37° C. A typical storage moduli of gels calculated using rheological assessment is depicted in FIG. 19. The general method was also used to assess the sheer-thinning properties of the gel which directly relates to their injectability (FIG. 18 (rheology), FIG. 13 (injectability)).

K) Environmental-scanning electron microscopy (ESEM): For ESEM analysis, an FEI Quanta 200 ESEM operating in ‘wet’ mode was used. The chamber is set at 100% Relative Humidity (peltier at 5 C and 6.2 Torr chamber pressure) at first and then the chamber pressure is decreased progressively to enhance surface detail (as the surface dried). The samples for ESEM are prepared at a concentration of 30 mg ml⁻¹.

L) Cell culture: Standard methodology is used to obtain cells. Once extracted and in the form of a pellet at the bottom of a tube, cells may be re-suspended in fresh medium and adjusted to the acquired cell concentrations before seeding on or into peptide hydrogels. Cells are counted using a haemocytometer at 1:1 mixture with trypan blue.

M) Live/dead assay: Cell viability is tested by a Live/dead assay (Invitrogen, UK). 1 ml of PBS containing 2.5 μl/ml of 4 μM ethidium homodimer-1 (EthD-1) assay solution and 1 μl/ml of 2 μM calcein AM assay solution is prepared. 300 μl of the live-dead solution is added on top of each sample for 15 minutes in the incubator at 37° C. in humidified atmosphere with 5% CO₂. The staining solution is removed and the samples are then viewed on a Leica SP5 confocal microscope or aNikon Eclipse E600 fluorescence microscope, with excitation filters of 494 nm (green, Calcein) and 528 nm (red, EthD-1). Images are captured using Lucia software. For quantitative analysis, a total of 250 cells are counted from each sample (mounted onto a glass slide using Prolong™ Gold anti-fade reagent (Invitrogen, UK)) over five randomly chosen areas and the viable and non-viable cells counts are recorded.

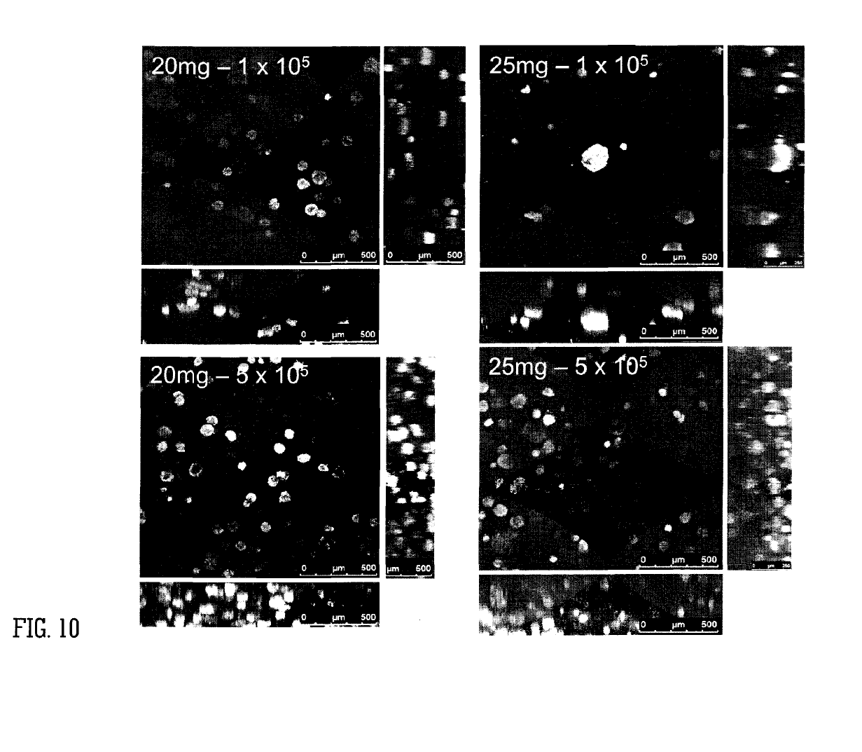

The above method was used to assess cell viability, as represented in FIGS. 10, 22-27.

N) LDH assay: The lactate dehydrogenase (LDH) assay (Promega, UK) is a colorimetric assay which measures an enzyme which is released upon cell lysis. It is used to quantify the number of viable cells in each gel. At each time interval, medium is removed from the wells, the gels transferred into microcentrifuge tubes and 500 μl fresh medium added. Samples are kept in the freezer at −80° C. for 30 minutes and then thawed for another 30 minutes at 37° C. The freeze-thaw cycles are carried out three times to lyse the cells. The microcentrifuge tubes are centrifuged at 250 g for 5 minutes and 50 μl of the supernatant was transferred to 96-well plate. 50 μl of the substrate mix was added. The well plates were incubated at room temperature. After 30 minutes the stop solution (containing acetic acid) is added to stop the reaction before the reading was taken. Readings in triplicate using a Lab Systems Ascent colorimetric plate reader with absorbance at 492 nm are recorded. Viable cell numbers are determined using the standard curve (average values normalised to a standard curve of known numbers of cultured cells i.e. 10⁶ to 10³ cells). Gels without cells serve as a negative control and the background absorbance is subtracted from the absorbance values recorded. Statistical analyses are performed using the One-way ANOVA test.

O) Cell count method: For each time point, medium is removed from on top of the samples (prepared in 24-well plates). 1 ml of fresh medium is added on top of each sample to dissolve the hydrogels. The mixture of the cell-gel suspension is transferred into 2 ml micro-centrifuge tubes. Vortex is applied gently to form a homogenous mixture. 50 μl of the cell suspension is transferred into a separate micro-centrifuge tube and diluted by adding 50 μl trypan blue. A haemocytometer is used to count viable cells accordingly.

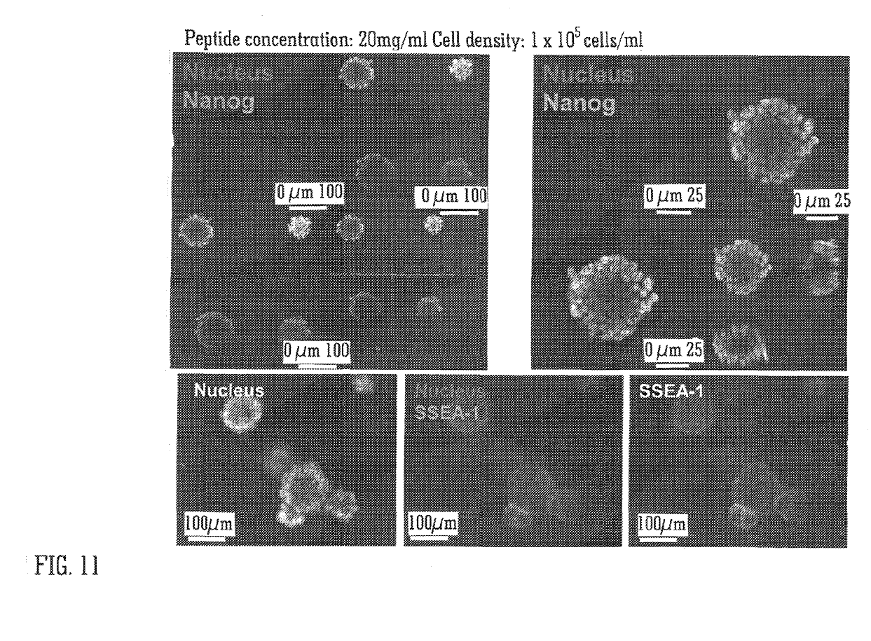

P) Immunocytochemistry staining: Gels are rinsed with PBS and fixed with 3% paraformaldehyde (PFA) for 15 minutes at room temperature. Fixation aims to preserve the shape of the cells and to stop all chemical reactions taking place inside the cells. Following fixation, the samples are washed using PBS before incubation with Immunocytochemistry (ICC) blocking buffer for 30 minutes. The ICC blocking buffer consists of 1% goat serum, 0.1% Triton X-100 and 50 mg/ml Bovine serum albumin (BSA). The goat serum and BSA reduces the non-specific binding and Triton X-100 is a detergent that permeabilises the cell membrane to allow larger molecules such as antibodies to enter the cells' interior. The samples are incubated in ICC block solution containing the primary antibody (rabbit polyclonal antibody to collagen type I/II purchased from Abeam, UK) for one hour at room temperature. To remove the non-bound primary antibody, samples are washed three times with PBS. Subsequently, TRITC conjugated secondary antibody (goat anti-rabbit IgG-Alexa Fluor® 594) is added for an hour in the dark. To remove the non-bound secondary antibody, samples are further washed five times with PBS. The gels are mounted onto the glass slides with Prolong™ Gold anti-fade reagent with DAPI (Invitrogen, UK). Images are captured using a fluorescence microscope (e.g. the Nikon Eclipse E600). FIG. 11 shows mES cells stained in a similar manner to that described above, using a different primary and secondary antibody combination.

Q) Collagen assay: The total collagen production in the scaffolds may be determined by the Sircol™ collagen assay kit (Biocolor, UK). Samples are hydrolysed in 24-well plates for 16 hours at 4° C. with pepsin concentration of 0.1 mg ml⁻¹ 0.5M acetic acid, pH 3. 1 ml of Sircol™ red reagent is added (on top) to 50 μl of each sample (adjusting the samples to a total volume of 100 μl a further 0.5M acetic acid) and mixed gently using a mechanical shaker for 30 minutes at room temperature. The collagen-dye complex is precipitated by centrifugation at 1200 rpm for 10 minutes, the supernatant is drained out and the collagen-dye pellet was dissolved in 1 ml of the alkali reagent. The mixture is vortexed and once all the bound dye has dissolved, 200 μl of each sample is transferred to individual wells of a 96-well plate. Absorbance measurements are taken at 555 nm wavelength, using a multi-well scanning spectrometer (Ascent microplate reader). Gel without cells serve as a negative control and the background absorbance is subtracted from the absorbance values recorded. The assay is performed in triplicate. Statistical analyses are performed using the One-way ANOVA test.

R) Statistical analysis: Statistical analysis may be performed using one-way ANOVA tests to indicate whether any difference in the mean cell levels (measured from the LDH assay and collagen assay) between the three test time points (day 1, day 14 and day 21) are significant. A significant F value is indicative of a significant difference in cell numbers between the three days where the probability is less than 0.05 (P<0.05).

S) Proliferation was also assessed using Click-iT® EdU Cell Proliferation Assays (Invitrogen catalogue number: C10340) according to the manufacturer's instructions. The above method was used to assess proliferation of hES cells in FIG. 21.

T) Printing a Microarray

The microarray used with the gel, allows very small spots of gel (30-60 nl) to be precisely placed on a microscope slides. It allows high throughput analysis of different gel/media/cell concentrations and also for drug screening applications. Gel spots containing viable human somatic cells have been successfully printed using a MicroSys 5100-4SQ noncontact microarray spotter (Genomics Solutions) and these were stable for three days when cultured in media.

For Gel Preparation:

Gel spots (here of 60 nl) containing cells were printed on to slides using a microarray printer (here a MicroSys 5100-4SQ noncontact microarray spotter). The method for creation up to step 1 (the first solution) was followed as described above. To encapsulate the cells, small aliquots of the first solution were mixed with cells in media, at a volume ratio of 2 parts media to 3 parts first solution.

The resultant arrays were set in a humidified chamber for at least 15 minutes at 37° C.

Media was then added to the surface in sufficient amount to ensure that the top of the gels were covered. Viable cells were visualised in the gels after three days of culture.

V) hES Cell Culture

We have used two human embryonic stem cell (hESC) lines (NCL-1 and HuES7) to demonstrate that the cells can be introduced into the gels and cultured within the gels, retaining cell viability. We have used two methods to do this, one with non-contact co-culture using a feeder layer and one with conditioned media.

Method 1 (Using NCL-1 as an Example):

The NCL-1 are grown in co-culture with mouse embryonic fibroblasts (MEFs) and manually subcultured every 5 days.

At least 24 hours prior to encapsulation, dishes of inactivated feeders are prepared in a 12 well tissue culture plate. On the day of encapsulation, MEF growth medium is removed from wells and replaced with 1 mL hES growth medium (KO-DMEM, 10% KOSR) containing ROCK inhibitor (10 μM) and incubated at 37° C., 5% CO² for at least one hour. Using a dissecting microscope in a laminar flow cabinet hESC cultures are assessed to identify undifferentiated colonies.

Using a cutting tool the colonies are scored, creating a grid, cutting the colony into 4-9 pieces.

The colony pieces are gently lifted from the feeder layer and placed into a 1.5 mL centrifuge tube containing 1 mL of hES growth media.

Centrifuge at 700 rpm for 3 minutes

Remove the media

[For single cell encapsulation, the media is removed and replaced with 500 μl of TrypLE™ and placed in a 37° C. water bath for 5 minutes.

The cells are centrifuged at 700 rpm for 3 minutes to remove the TrypLE™.]

The cells are resuspended in 250 μL of hES growth medium and add to the gels following established protocol (as already detailed for mouse ES cells). 300 μL of gel is plated into each transwell insert.

After 4 media washes are completed, the transwell insert is placed into the 12 well tissue culture plate containing the inactivated feeders, 50 μL of hES growth media is gently added into the top of the insert.

The cells are fed with fresh growth medium 2 days after encapsulation by replacing the media in culture well. The media is not changed on top of the well.

Continue to feed every day.

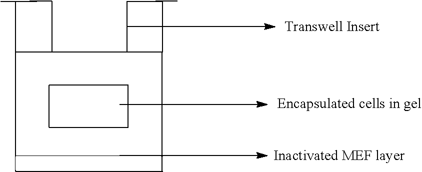

Method 2 (Conditioned Media)

At least 48 hours prior to encapsulation, dishes of inactivated feeders are prepared in a 12 well tissue culture plate.

24 hours after plating the MEF growth medium is removed from wells and replaced with 1 mL hES growth medium containing ROCK inhibitor (10 μM) This is incubated at 37° C., 5% CO² for 24 hours. (This will now constitute the conditioned hES growth media).

Using a dissecting microscope in a laminar flow cabinet hESC cultures assessed to identify undifferentiated colonies.

Using a cutting tool the colonies are scored, creating a grid, cutting the colony into 4-9 pieces.

The colony pieces are gently lifted from the feeder layer and placed into a 1.5 mL centrifuge tube containing 1 mL of hES growth media.

The cells are centrifuged at 700 rpm for 3 minutes and the media removed.

[For single cell encapsulation, the media is removed and replaced with 500 μl of TrypLE™ and placed in a 37° C. water bath for 5 minutes.

The cells are centrifuged at 700 rpm for 3 minutes to remove the TrypLE™.]

Cells are resuspended in 250 μL of hES growth medium and added to the gels following established protocol (as for mouse ES cells). 300 μL of gel is plated into each transwell insert.

After completing 4 media washes the transwell insert is placed into a 12 well tissue culture plate.

950 μL of the conditioned hES growth media is added to each well. 50 μL of conditioned hES growth media is gently added into the top of the transwell.

The media on the MEFs is replaced with 1 mL of hES growth media and continue to incubate at 37° C., 5% CO².

Every 2 days after encapsulation continue to feed cells with conditioned hES media by replacing the media in culture well. The media is not changed on top of the well.

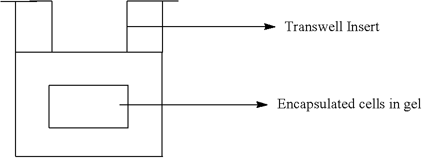

### APPENDIX

MEF seeding density

Inactivated MEFs should be seeded at a density of 1.4× 104 and used within 5 days.

MEF media

DMEM—Gibco—41966052

10% FBS

2 mM L-Glutamine—Gibco 25030-24

NCL-1 hES media

Knockout DMEM media—Gibco 10829-018

10% Knockout Serum Replacement—Gibco 10828-028

1 mM L-Glutamine—Gibco 25030-24

100 mM Non Essential Amino Acids—Gibco 11140-035

0.1 mM β mercaptoethanol—Gibco 31350-010

8 ng/mL human bFGF

Transwell—Millipore PIRP15R48, Millicell Hanging Cell Culture Insert, PET 1.0 uM

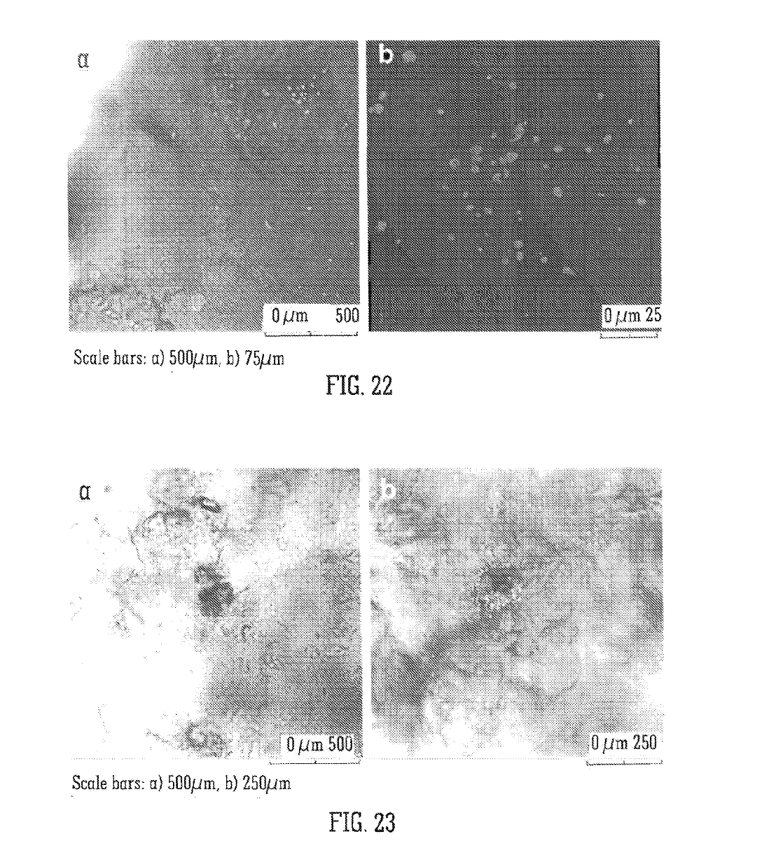

The above method was used to encapsulate human ES cells (FIG. 20) supporting proliferation (FIG. 21) and viable cells/aggregates (FIGS. 22-25).

1-49. (canceled)

50. A method of making a hydrogel, comprising the steps of:
1) providing a peptide solution, wherein the peptide comprises an amphiphilic portion;
2) altering a characteristic of the peptide solution, wherein the characteristic is ionic strength, pH, temperature, or ion concentration, to form an optically transparent first solution;
3) maintaining the solution of 2) under conditions suitable for the formation of a hydrogel;
4) liquefying the hydrogel of 3), preferably by mechanical agitation;
5) combining the liquefied hydrogel of 4) with i) cells and optionally ii) media to form a second solution and optionally determining the optical transparency of the second solution, to assess its capability of forming a hydrogel; and
6) maintaining the second solution of 5) under conditions suitable for the formation of a hydrogel comprising cells encapsulated therein.

51. A method of making a hydrogel according to claim 50, wherein the characteristic of step 2) to be altered is the pH, wherein preferably the alteration comprises increasing the pH to between 8 and 10, preferably to between 9 and 9.5, preferably to form a homogenous first liquid, preferably at a temperature between 60° C. and 95° C.

52. A method of making a hydrogel according to claim 50, wherein step 1) and/or 2) further comprises the step of adding a buffer solution to either the peptide solution or first solution, preferably wherein where the buffer is added to the first solution it is added at a ratio of 1 part buffer to between 2 and 8 parts first solution; and preferably wherein the buffer may be selected from the group consisting of PBS, Hank's balanced salt solution, and Earle's balanced salt solution.

53. A method of making a hydrogel according to claim 50, wherein step 5) comprises adding cells with media wherein the media is an aqueous media, preferably selected from the group consisting of DMEM, KO-DMEM, Iscove's, Modified Dulbecco's media, Stem Pro, mTeSR, water, saline or serum; and
wherein preferably step 5) comprises the steps of:
calculating the amount of media for addition to the liquefied gel of step 4) to provide media:liquefied gel ratio of between 1:1 and 1:10, preferably between 1:2 and 1:5 more preferably 1:4;
ii. mixing media with the liquefied gel until an optically transparent second solution is formed.

54. A method of making a hydrogel according to claim 50, wherein conditions suitable for formation into a hydrogel are between 20° C. and 37° C.

55. A method of making a hydrogel according to claim 50, further comprising storing the hydrogel formed in step 3) at about 4° C.; and/or freezing the hydrogel formed in step 3).

56. A method of making a hydrogel according to claim 50, further comprising the steps of:
i) determining the final volume of the gel to be made;
ii) determining the final concentration of the gel;
iii) calculating the amount of peptide to be used as volume (m/1) and final concentration;
iv) mixing the peptide of ii) or iii) with the base to produce a first solution;
v) determining the optical transparency of the first solution;
vi) if the first solution is not optically transparent, continuing to add base until an optically transparent solution is obtained;
vii) maintaining the solution under conditions suitable for formation of a hydrogel;
viii) assessing gel stability; wherein if unstable, discarding and repeating steps i) to vi) wherein a reduced amount of base is added in step iv);
xi) calculating the total amount of base added per mg of peptide used to obtain an optically transparent first solution and a stable hydrogel;
preferably wherein
i) where the solution is not optically transparent or the gel is not stable, increasing the amount of base in step iii) by between 0.3 and 10% of the final gel volume until an optically transparent solution is obtained;
ii) where the first solution is optically transparent but the gel unstable, reducing the amount of base in step iii) until the gel is stable.

57. A method of encapsulating cells in a hydrogel, comprising:
1) providing a hydrogel that has been pre-formed or has been previously frozen and defrosted, and/or stored at about 4° C.;
2) liquefying the hydrogel;
3) combining the liquefied hydrogel of 2) with i) cells and optionally i) media to form a second solution;
4) maintaining the second solution of 3) under conditions suitable for the formation of a hydrogel;
preferably wherein the pre-formed hydrogel is prepared by steps 1) to 3) of claim 50.

58. A method of culturing cells, the method comprising:
1) encapsulating cells in a hydrogel, according to a method of claim 57;
2) maintaining the hydrogel under biologically acceptable cell culture conditions for a cell culture period;
3) optionally washing the hydrogel during the cell culture period, preferably using an aqueous media, preferably selected from the group consisting of DMEM, KO-DMDM, Iscove's Modified Dulbecco's media, Stem Pro, mTeSR, water, saline or serum.

59. A method of extracting a cell and/or cell-derived material from a hydrogel, the method comprising:
1) disrupting a hydrogel comprising cells prepared according to claim 50, to fully or partially liquefy the gel, wherein the disruption is preferably performed mechanically;
2) separating any cells from the hydrogel or liquefied hydrogel;
3) optionally washing the cells; and optionally either:
i) extracting nucleic acids including RNA or DNA, proteins, sugars or other cellular material from the cell; or
ii) passaging an extracted cell comprising i) mixing an extracted cell with media; ii) introducing the media comprising a cell into a second or further hydrogel.

60. A hydrogel for use in a method of tissue repair in a subject wherein the method comprises:
i) liquefying a hydrogel containing a cell, wherein the hydrogel is prepared according to claim 50; and
ii) introducing the liquefied hydrogel comprising a cell of i) into an appropriate tissue of a subject.

61. A method of printing a microarray, comprising making a hydrogel according to claim 50, and prior to step 6), spotting the gel onto an array.

62. A hydrogel which has one or more of the following independently selected properties:
i) is capable of a liquid-gel-liquid transition or a gel-liquid-gel transition
ii) is capable of maintaining cells in culture for at least 1 hour, 2, 3, 4, 5, l 6, 7, 8, 9, 10, 12, 15, 20 or 24 hours or at least 1, 2, 3, 4, 5, 6, 7, 8, 9, 10, 11, 12, 13, 14, 21, 25, 28, or 31 days, preferably in 3D;
iii) is capable of supporting proliferation of cells, preferably in 3D;
iv) is capable of enabling extraction of viable cells;
v) is capable of supporting transition of cells to a second or further hydrogel;
vi) is capable of maintaining a three dimensional structure;
vii) comprises nanofibers of between 2 and 20 nm in width; and
viii) exhibits stability for at least 1, 2, 3, 4, 5, 6, 7, 8, 9, 10, 11, 12, 13, 14, 21, 25, 28 or 31 days;
x) is made by a method of claim 50;
and preferably comprises at least 80% (w/w) water.

63. A kit for making a hydrogel according to a method of claim 50, wherein the kit comprises either dry peptide, peptide solution or peptide gel, preferably a peptide gel, and instructions for carrying out a method of the invention; and optionally one or more of base, buffer, media, cell, a vessel, and means for introducing the gel into a subject.

64. A method according to claim 50 wherein a) the amphiphilic portion is i) between 2 and 20 amino acids, preferably 8 amino acids in length; ii) comprises the sequence FEFE [SEQ ID NO: 3] and/or FKFK [SEQ ID NO: 4], and preferably the sequence FEFEFKFK [SEQ ID NO: 1]; and/or b) the peptide comprises a non-amphiphilic portion, for example a binding site, cross-linking site, or a sequence for functionalising the peptide and preferably wherein the final peptide concentration of the hydrogel is at or above the Critical Gelation Concentration, preferably between 15 and 40 mg/ml, more preferably between 20 and 25 mg/ml.

65. A hydrogel according to claim 62 wherein a) the amphiphilic portion is i) between 2 and 20 amino acids, preferably 8 amino acids in length; ii) comprises the sequence FEFE [SEQ ID NO: 3] and/or FKFK [SEQ ID NO: 4], and preferably the sequence FEFEFKFK [SEQ ID NO: 1]; and/or b) the peptide comprises a non-amphiphilic portion, for example a binding site, cross-linking site, or a sequence for functionalising the peptide and preferably wherein the final peptide concentration of the hydrogel is at or above the Critical Gelation Concentration, preferably between 15 and 40 mg/ml, more preferably between 20 and 25 mg/ml.

66. A kit according to claim 63 wherein a) the amphiphilic portion is i) between 2 and 20 amino acids, preferably 8 amino acids in length; ii) comprises the sequence FEFE [SEQ ID NO: 3] and/or FKFK [SEQ ID NO: 4], and preferably the sequence FEFEFKFK [SEQ ID NO: 1]; and/or b) the peptide comprises a non-amphiphilic portion, for example a binding site, cross-linking site, or a sequence for functionalising the peptide and preferably wherein the final peptide concentration of the hydrogel is at or above the Critical Gelation Concentration, preferably between 15 and 40 mg/ml, more preferably between 20 and 25 mg/ml.

67. A method according to claim 59, wherein ii) introducing the media comprising a cell into a second or further hydrogel comprises a method of encapsulating cells in a hydrogel, comprising:
1) providing a hydrogel that has been pre-formed or has been previously frozen and defrosted, and/or stored at about 4° C.;
2) liquefying the hydrogel;
3) combining the liquefied hydrogel of 2) with i) cells and optionally ii) media to form a second solution;
4) maintaining the second solution of 3) under conditions suitable for the formation of a hydrogel.

68. A method according to claim 67, wherein the pre-formed hydrogel is prepared by
1) providing a peptide solution, wherein the peptide comprises an amphiphilic portion;
2) altering a characteristic of the peptide solution, wherein the characteristic is ionic strength, pH, temperature, or ion concentration, to form an optically transparent first solution; and
3) maintaining the solution of 2) under conditions suitable for the formation of a hydrogel.


SEQUENCE LISTING (7 sequences)

SEQ ID NO: 1 - protein, 8 aa, Artificial Sequence
  PEPTIDE
FEFEFKFK

SEQ ID NO: 2 - protein, 8 aa, Artificial Sequence
  PEPTIDE
VEVEVKVK

SEQ ID NO: 3 - protein, 4 aa, Artificial Sequence
  PEPTIDE
FEFE

SEQ ID NO: 4 - protein, 4 aa, Artificial Sequence
  PEPTIDE
FKFK

SEQ ID NO: 5 - protein, 8 aa, Artificial Sequence
  PEPTIDE
FEFKFEFK

SEQ ID NO: 6 - protein, 8 aa, Artificial Sequence
  PEPTIDE
LELELKLK

SEQ ID NO: 7 - protein, 8 aa, Artificial Sequence
  PEPTIDE
VEVKVEVK